(19) United States
(12) Patent Application Publication
(10) Pub. No.: US 2024/0415414 A1
(43) Pub. Date: Dec. 19, 2024

(54) SYSTEMS, DEVICES, AND METHODS FOR ANALYTE MONITORING

(71) Applicant: ABBOTT DIABETES CARE INC., Alameda, CA (US)

(72) Inventors: Peter G. Robinson, Alamo, CA (US); Dharmendra S. Patel, Witney (GB); Peter M. Voit, Dublin, CA (US); Vivek S. Rao, Alameda, CA (US); Jennifer Olson, Alameda, CA (US); Shridhara A. Karinka, Pleasanton, CA (US); Leilani L. Berchasi, Alameda, CA (US); Jean-Pierre Cole, Tracy, CA (US); Sebastien Doan Ky Nam Nguyen, Novato, CA (US); Xuandong Hua, Mountain View, CA (US); Mahmood Reza Kazemi, Alameda, CA (US); Stephen T. Pudjijanto, San Ramon, CA (US)

(21) Appl. No.: 18/667,158

(22) Filed: May 17, 2024

Related U.S. Application Data
(60) Provisional application No. 63/467,573, filed on May 18, 2023.

(51) Int. Cl. A61B 5/145

(52) U.S. Cl. CPC A61B 5/14503; A61B 5/1451; A61B 2560/063; A61B 2562/04

(57) ABSTRACT

Systems, devices and methods are provided for inserting at least a portion of an in vivo analyte sensor for sensing an analyte level in a bodily fluid of a subject. In particular, disclosed herein are various embodiments of sensor control devices, and components thereof, designed to reduce the size and the number of internal components of the sensor control device. Further, the embodiments of the sensor control device and related sensor features disclosed herein are designed to increase comfort and convenience for the subject.

### CROSS-REFERENCE TO RELATED APPLICATIONS

The application claims priority to U.S. Application Ser. No. 63/467,573 filed May 18, 2023, which is hereby expressly incorporated by reference in its entirety for all purposes.

### FIELD

The subject matter described herein relates generally to systems, devices, and methods for in vivo analyte monitoring.

### BACKGROUND

The detection and/or monitoring of analyte levels, such as glucose, ketones, lactate, oxygen, hemoglobin AIC, or the like, can be vitally important to the overall health of a person, particularly for an individual having diabetes. Patients suffering from diabetes mellitus can experience complications including loss of consciousness, cardiovascular disease, retinopathy, neuropathy, and nephropathy Persons with diabetes are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor, and can be assembled and applied by the individual with a sensor applicator. The application process includes inserting a sensor, such as an analyte sensor that senses a user's analyte level in a bodily fluid, using an applicator or insertion mechanism, such that the sensor comes into contact with a bodily fluid. The sensor control device may also be configured to transmit analyte data to another device, from which the individual or her health care provider (“HCP”) can review the data and make therapy decisions.

While current sensors can be convenient for users, they can be made more comfortable, convenient, and portable by further reducing the size of the sensor control device. Furthermore, by reducing the size of the sensor control device, and/or by reducing the number of internal components, the manufacturing cost of the sensor control device can be reduced. Lower manufacturing costs can be one means of reducing replacement costs for a patient, since the on-body unit can be a disposable, one-time use unit which needs regular replacement. One limit to such miniaturization is the need for a sensor substrate for the locating electrodes for sensing analyte concentration and separate substrate for locating electronic components for providing electrical power, processing sensor data, and transmitting sensor data to a remote device. However, previous manufacturing technologies prevent such components from being mounted directly to the sensor substrate.

Thus, a need exists for a continuous analyte monitoring system which has a reduced size, provides discreet monitoring to encourage frequent analyte monitoring to improve glycemic control, and is economical to manufacture.

### SUMMARY

Systems, devices and methods are provided for inserting at least a portion of an in vivo analyte sensor for sensing an analyte level in a bodily fluid of a subject. In particular, disclosed herein are various embodiments of sensor control devices, and components thereof, designed to reduce the size and the number of internal components of the sensor control device. Further, the embodiments of the sensor control device and related sensor features disclosed herein are designed to increase comfort and convenience for the subject. Provided herein are example embodiments of systems, devices and methods for the assembly and use of an applicator and a sensor control device of an in vivo analyte monitoring system. A sensor control device can be provided which comprises an analyte sensor (herein also referred to as a “sensor”) integrated with sensor electronics. According to some embodiments, the analyte sensor comprises an in vivo portion and an ex vivo portion. The in vivo portion is configured to be positioned in contact with an interstitial fluid of a user and to generate signals associated with a measured analyte level. The ex vivo portion can comprise a plurality of sensor electronics mounted thereon. In some embodiments, the sensor electronics can include one or more processors, one or more batteries, an antenna, a semiconductor chip, to name a few.

As embodied herein, the in vivo portion can include a substrate, at least one working electrode, and a reference electrode. The plurality of sensor electronics on the ex vivo portion can be communicatively coupled to the at least one working electrode and reference electrode, and can be configured to receive signals associated with the measured analyte level generated by the electrodes. As embodied herein, the analyte sensor can be configured to sense at least one of lactate, glucose, or ketone.

According to an aspect of the embodiments, the sensor electronics can include a battery having a first tab and a second tab, wherein the tabs are oriented radially from one another. In some embodiments, the first tab and second tab are coplanar. In other embodiments, the first tab and second are non-coplanar. In some embodiments, the first tab and second tab are parallel and symmetric to one another. In some embodiments, the first tab is welded on a positively charged surface of the battery so as to further reduce height of the sensor control device.

According to another aspect of the embodiments, at least a portion of the ex vivo portion of the analyte sensor is folded such that the size of the sensor control device is further reduced.

In some embodiments, a sharp and sharp carrier are utilized to facilitate insertion of the analyte sensor under the skin surface of the user. In some embodiments, the sharp can comprise a cantilever arm configured to engage with or snap into a corresponding portion of the sharp carrier. In some embodiments, the sharp can comprise a sharp window configured to engage with or snap into a corresponding portion of the sharp carrier.

According to another aspect of the embodiments, the sharp carrier comprises an inner portion and an outer portion, wherein the inner portion comprises a cavity configured for receipt of at least a portion of the sharp (e.g., an upper portion of the sharp). The inner portion of the sharp carrier further comprises a sharp channel on a distal surface thereof so as to allow the upper portion of the sharp to extend therethrough and into the cavity. In some embodiments, upper portion of the sharp is positioned proximal relative to an upper portion of the sensor control device. Further, the lower portion is configured to extend through an aperture in the upper portion and a mount portion of the sensor control device. Specifically, the aperture extending through the upper portion and mount portion aligns with a hole on an adhesive patch which is distal relative to the mount. In some embodiments, at least a portion of the sharp is arranged adjacent to the analyte sensor. In some embodiments, the sharp comprises a distal tip which can penetrate the skin while carrying the in vivo portion of the analyte sensor so as to facilitate contact of the analyte sensor with bodily fluid of the user.

According to some embodiments, the analyte sensor and the one or more sensor electronics are formed from a one-piece substrate. In some embodiments, the substrate can be formed through a die-cutting process, a laser cutting process, an ultrasonic cutting process, a molding process, a stamping process, or a 3-D printing process. The substrate can be made of a flexible non-electrically-conductive polymer. In some embodiments, the substrate is a polyamide substrate, a polyester substrate, or a polyethylene terephthalate substrate.

According to another aspect of the embodiments, a sensor control device can be provided which is partially or entirely flexible. In some embodiments, the sensor control device can be “band-aid” shape or be a flexible strip with one or more rounded edges. In some embodiments, the sensor control device houses the sensor electronics within a structurally rigid portion. In some embodiments, the sensor electronics include one or more printed batteries that are configured to be stacked or layered and are flexible. In some embodiments, the sensor control device can include an array of analyte sensors comprising a shortened sensor tail with a sharpened tip portion. The shortened sensor tail can be any length needed to reach an interstitial fluid of the user. In some embodiments, the shortened sensor tail can be between 0.8 millimeters to three millimeters in length. In some embodiments, each of the analyte sensors in the array can include a different chemistry on the sensor tail. In some embodiments, the sensor tails comprise an enteric coating that is configured to dissolve after a portion of the analyte sensor (e.g., the sensor tail) has been inserted into the skin of the user.

According to another aspect of the embodiments, an applicator comprising a handle and a dispensing portion configured to receive a dispenser is utilized for application of the sensor control device onto the skin surface of the user. In some embodiments, the applicator utilizes a roll-out application process or a “push and roll” mechanism. In some embodiments, the user must exert a downward pressure to initiate the roll-out application process. A minimum insertion pressure can be achieved by ensuring that dispensing or rolling does not activate until a predetermined amount of pressure is manually applied by the user. Once the application process has been initiated by the minimum insertion pressure being achieved, the user can then dispense or roll the dispenser so as to apply the sensor control device onto the user's skin surface.

The embodiments provided herein are improvements to reduce the size and number of internal components of the sensor control device. Further the embodiments provided herein are improvements to reduce costs and provide for a more convenient, comfortable, and portable sensor control device. Other improvements and advantages are provided as well. The various configurations of these devices are described in detail by way of embodiments which are only examples.

Other systems, devices, methods, features and advantages of the subject matter described herein will be or will become apparent to one with skill in the art upon examination of the following figures and detailed description. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a sensor applicator, reader device, monitoring system, network, and remote system.

FIG. 2A is a block diagram depicting an example embodiment of a reader device.

FIGS. 2B and 2C are block diagrams depicting example embodiments of sensor control devices.

FIG. 3A is a side view depicting an example embodiment of an applicator device coupled with a cap.

FIG. 3B is a side perspective view depicting an example embodiment of an applicator device and cap decoupled.

FIG. 3C is a perspective view depicting an example embodiment of a distal end of an applicator device and electronics housing.

FIG. 4A is side view depicting an example embodiment of a housing.

FIG. 4B is a perspective view depicting an example embodiment of a distal end of a housing.

FIG. 4C is a side cross-sectional view depicting an example embodiment of a housing.

FIG. 5A is a side view depicting an example embodiment of a sheath.

FIG. 5B is a perspective view depicting an example embodiment of a proximal end of a sheath.

FIG. 5C is a close-up perspective view depicting an example embodiment of a distal side of a detent snap of a sheath.

FIG. 5D is a side view depicting an example embodiment of features of a sheath.

FIG. 5E is an end view of an example embodiment of a proximal end of a sheath.

FIG. 6A is a proximal perspective view depicting an example embodiment of a device carrier.

FIG. 6B is a distal perspective view depicting an example embodiment of a device carrier.

FIG. 7 is a proximal perspective view of an example embodiment of a sharp carrier.

FIG. 8 is a side cross-section depicting an example embodiment of a sharp carrier.

FIG. 9A is a top exploded view of an example embodiment of a sensor control device.

FIG. 9B is a bottom exploded view of an example embodiment of a sensor control device.

FIGS. 10A-1 to 10A-3 are top perspective views of exemplar embodiments of sensor control devices.

FIGS. 10B-I to 10B-3 are perspective views of exemplar embodiments of sensor control devices.

FIGS. 10C-1 to 10C-3 are side views of exemplar embodiments of sensor control devices.

FIGS. 11A-1 and 11A-2 are top and bottom perspective views, respectively, of an exemplar embodiment of a battery.

FIGS. 11B-1 and 11B-2 are top and bottom perspective views, respectively, of an exemplar embodiment of a battery.

FIGS. 12A-1 to 12A-3 are top perspective views of an exemplar embodiment of an analyte sensor with integrated sensor electronics.

FIG. 12A-4 is a bottom perspective view of an exemplar embodiment of an analyte sensor with integrated sensor electronics.

FIGS. 12B-I to 12B-3 are top perspective views of an exemplar embodiment of an analyte sensor with integrated sensor electronics.

FIGS. 13A and 13B are side views depicting an exemplar embodiment of a sensor control device.

FIG. 14 is an exploded view of an exemplar embodiment of a sensor control device.

FIG. 15 is a perspective view of an exemplar embodiment of a sharp.

FIG. 16 is a cutaway view depicting an exemplar embodiment of a sharp carrier operatively coupled with a sharp and sensor control device.

FIGS. 17A-17E illustrate cross-sectional views depicting an example embodiment of an applicator during various stages of deployment.

FIGS. 18A and 18B are top and bottom perspective views, respectively, of an example embodiment of a sensor control device.

FIG. 18C is a side perspective view of an example embodiment of a sensor control device.

FIG. 18D is a top perspective view of an example embodiment of a sensor control device.

FIG. 18E is a bottom perspective view of an example embodiment of a sensor control device.

FIG. 18F is a bottom perspective view of an example embodiment of a sensor control device.

FIG. 18G is a top perspective view of an example embodiment of a sensor control device.

FIG. 18H is a bottom perspective view of an example embodiment of a sensor control device.

FIG. 19 is a perspective view of an example embodiment of an applicator with a dispenser.

FIGS. 20A, 20B, and 20C are side perspective, back side, and bottom side views, respectively, of an example embodiment of an applicator.

FIGS. 21A and 21B are top side perspective and side views, respectively, of an example embodiment of a dispenser.

FIG. 21C is a top side perspective view of an example embodiment of an inner ring of a dispenser.

FIG. 21D is a side perspective view of an example embodiment of a dispenser loaded with a sensor control device.

FIGS. 22A and 22B depict various stages of a roll-out application process to deploy a sensor control device.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include systems, devices, and methods for the use of analyte sensor insertion applicators for use with in vivo analyte monitoring systems. An applicator can be used to position the sensor control device on a human body with an analyte sensor in contact with the wearer's bodily fluid. The embodiments provided herein are improvements to reduce the likelihood that a sensor is improperly inserted or damaged, or elicits an adverse physiological response. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Exemplary In Vivo Analyte Monitoring System

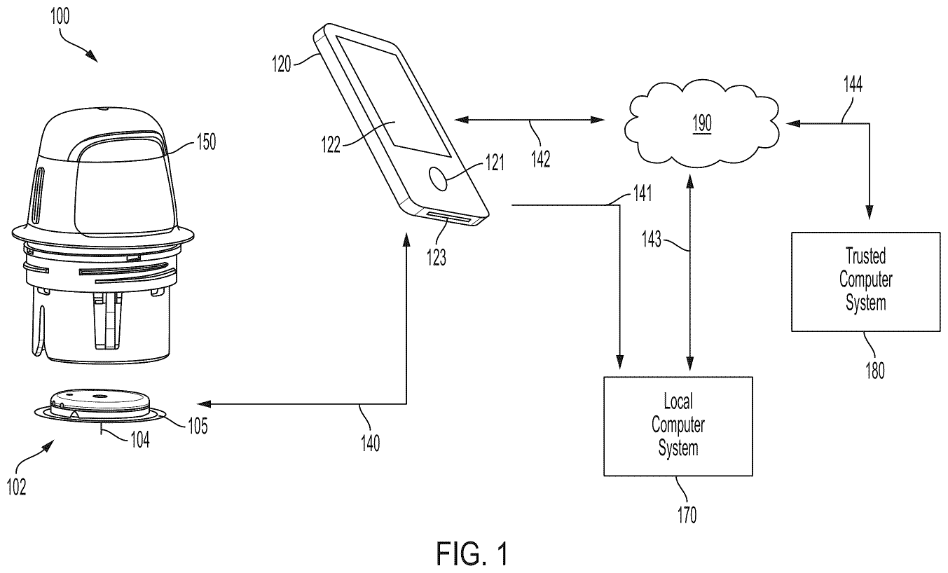

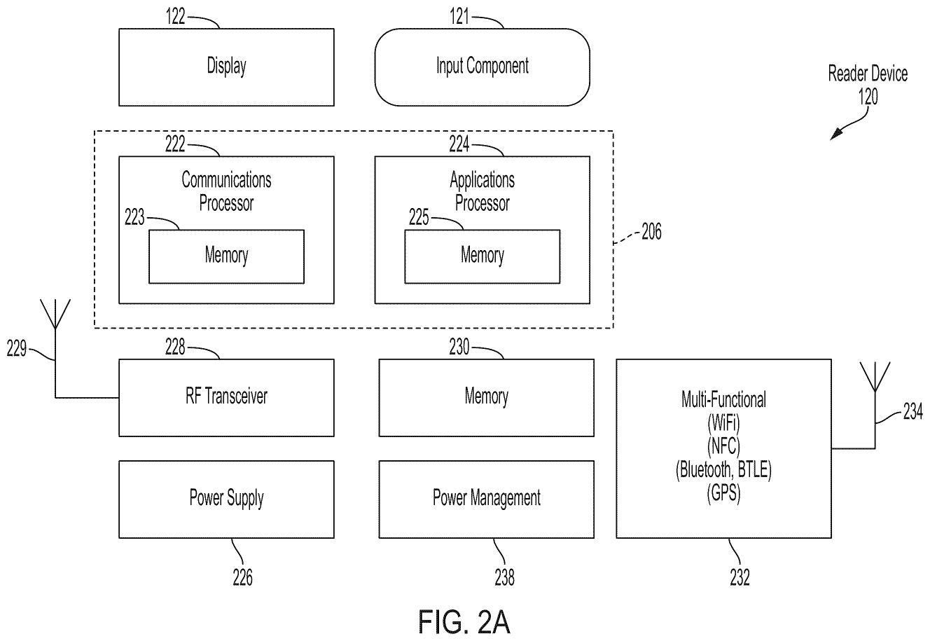

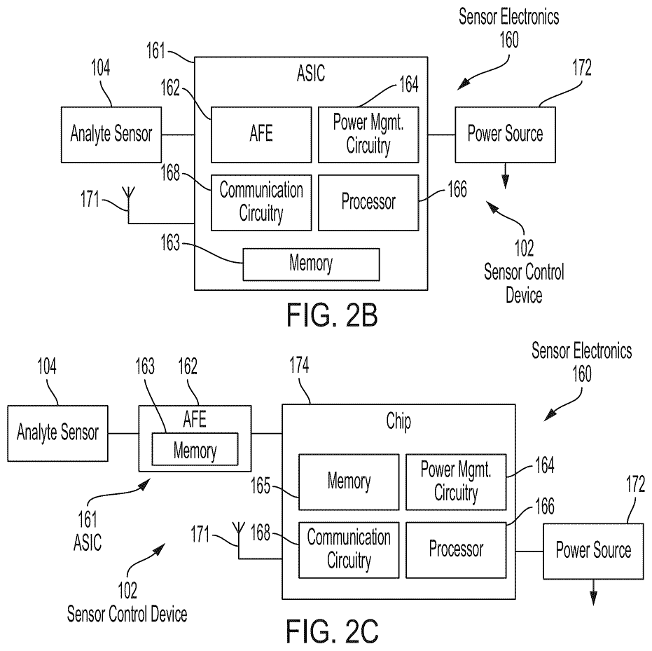

FIG. 1 is a conceptual diagram depicting an example embodiment of an analyte monitoring system 100 that includes a sensor applicator 150, a sensor control device 102, and a reader device 120. Sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on reader device 120 using screen 122 and input 121 and the device battery can be recharged using power port 123. More detail about reader device 120 is set forth with respect to FIG. 2A below. Reader device 120 can communicate with local computer system 170 via a communication path 141 using a wired or wireless technique. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy, Wi-Fi or others. Local computer system 170 can communicate via communications path 143 with a network 190 similar to how reader device 120 can communicate via a communications path 142 with network 190, by wired or wireless technique as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

### Exemplary Reader Device

FIG. 2A is a block diagram depicting an example embodiment of a reader device configured as a smartphone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further included can be a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

### Exemplary Sensor Control Devices

FIGS. 2B and 2C are block diagrams depicting example embodiments of sensor control device 102 having analyte sensor 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiment of Sensor Applicator Device

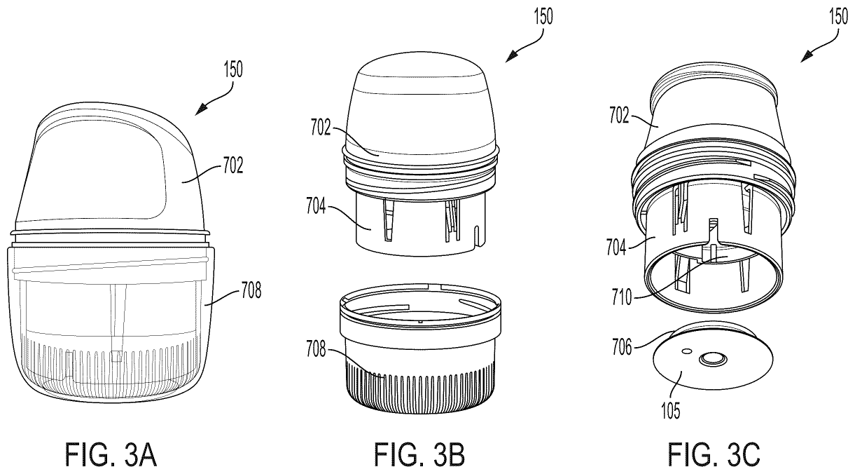

FIG. 3A is a side view depicting an example embodiment of an applicator device 150 coupled with screw cap 708. This is one example of how applicator 150 is shipped to and received by a user, prior to assembly by the user with a sensor. In other embodiments, applicator 150 can be shipped to the user with the sensor and sharp contained therein. FIG. 3B is a side perspective view depicting applicator 150 and cap 708 after being decoupled. FIG. 3C is a perspective view depicting an example embodiment of a distal end of an applicator device 150 with electronics housing 706 and adhesive patch 105 removed from the position they would have retained within device carrier 710 of sheath 704, when cap 708 is in place.

### Example Embodiment of Applicator Housing

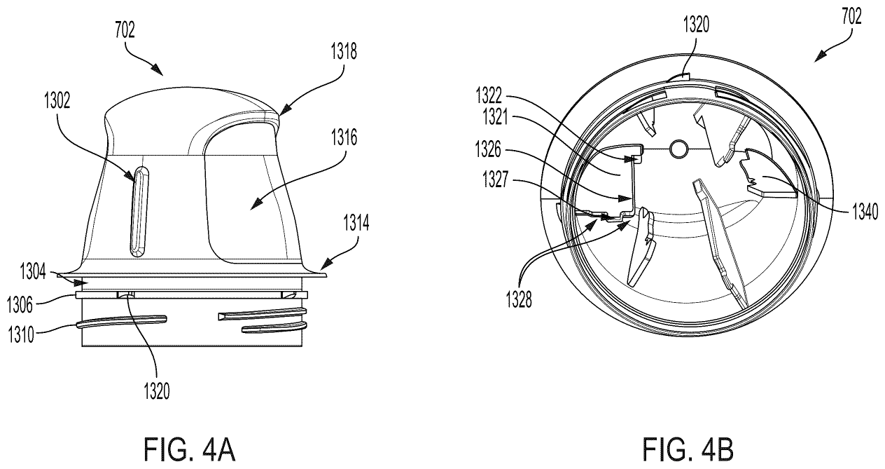

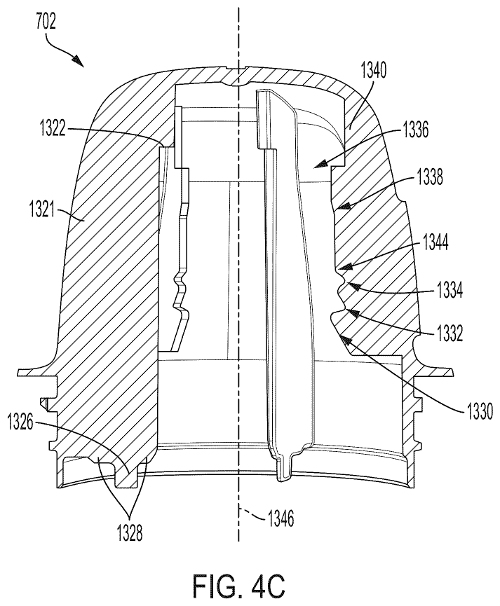

FIG. 4A is side view depicting an example embodiment of the applicator housing 702 that can include an internal cavity with support structures for applicator function. A user can push housing 702 in a distal direction to activate the applicator assembly process and then also to cause delivery of sensor control device 102, after which the cavity of housing 702 can act as a receptacle for a sharp. In the example embodiment, various features are shown including housing orienting feature 1302 for orienting the device during assembly and use. Tamper ring groove 1304 can be a recess located around an outer circumference of housing 702, distal to a tamper ring protector 1314 and proximal to a tamper ring retainer 1306. Tamper ring groove 1304 can retain a tamper ring so users can identify whether the device has been tampered with or otherwise used. Housing threads 1310 can secure housing 702 to complimentary threads on cap 708 (FIGS. 3A and 3B) by aligning with complimentary cap threads and rotating in a clockwise or counterclockwise direction. A side grip zone 1316 of housing 702 can provide an exterior surface location where a user can grip housing 702 in order to use it. Grip overhang 1318 is a slightly raised ridge with respect to side grip zone 1316 which can aid in ease of removal of housing 702 from cap 708. A shark tooth 1320 can be a raised section with a flat side located on a clockwise edge to shear off a tamper ring (not shown), and hold tamper ring in place after a user has unscrewed cap 708 and housing 702. In the example embodiment four shark teeth 1320 are used, although more or less can be used as desired.

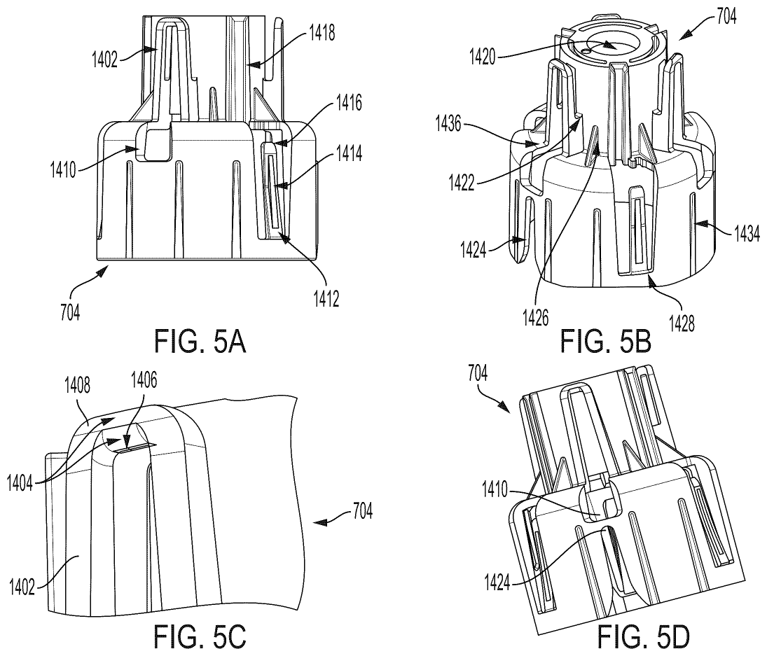

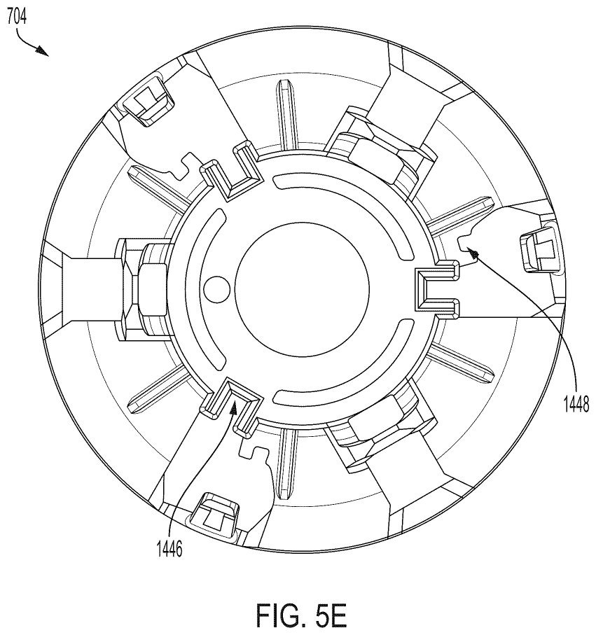

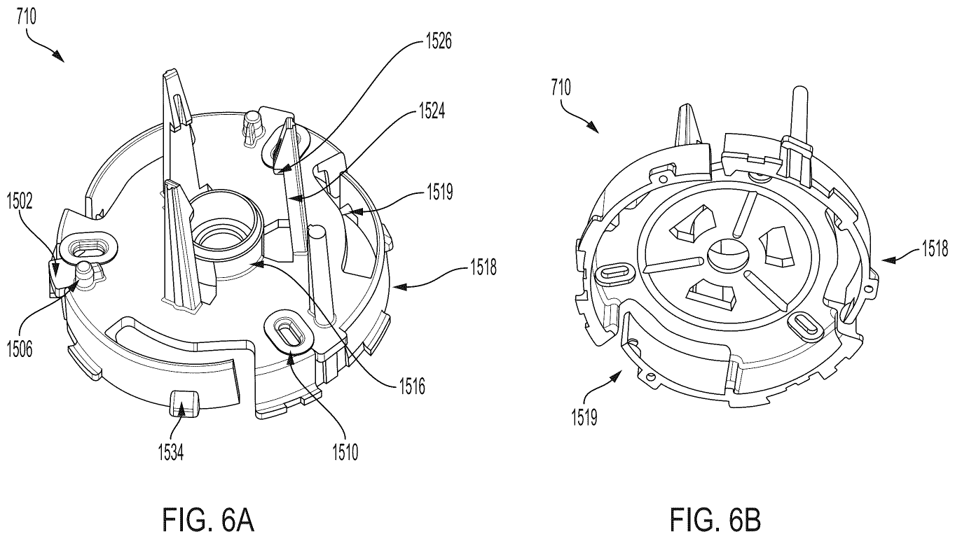

FIG. 4B is a perspective view depicting a distal end of housing 702. Here, three housing guide structures (or “guide ribs”) 1321 are located at 120 degree angles with respect to each other, and at 60 degree angles with respect to locking structures (or “locking ribs”) 1340, of which there are also three at 120 degree angles with respect to each other. Other angular orientations, either symmetric or asymmetric, can be used, as well as any number of one or more structures 1321 and 1340. Here, each structure 1321 and 1340 is configured as a planar rib, although other shapes can be used. Each guide rib 1321 includes a guide edge (also called a “sheath guide rail”) 1326 that can pass along a surface of sheath 704 (e.g., guide rail 1418 described with respect to FIG. 5A). An insertion hard stop 1322 can be a flat, distally facing surface of housing guide rib 1321 located near a proximal end of housing guide rib 1321. Insertion hard stop 1322 provides a surface for a sensor electronics carrier travel limiter face 1420 of a sheath 704 (FIG. 5B) to abut during use, preventing sensor electronics carrier travel limiter face 1420 from moving any further in a proximal direction. A carrier interface post 1327 passes through an aperture 1510 (FIG. 6A) of device carrier 710 during an assembly. A device carrier interface 1328 can be a rounded, distally facing surface of housing guide ribs 1321 which interfaces with device carrier 710.

FIG. 4C is a side cross-section depicting an example embodiment of a housing. In the example embodiment, side cross-sectional profiles of housing guide rib 1321 and locking rib 1340 are shown. Locking rib 1340 includes sheath snap lead-in feature 1330 near a distal end of locking rib 1340 which flares outward from central axis 1346 of housing 702 distally. Each sheath snap lead-in feature 1330 causes detent snap round 1404 of detent snap 1402 of sheath 704 as shown in FIG. 5C to bend inward toward central axis 1346 as sheath 704 moves towards the proximal end of housing 702. Once past a distal point of sheath snap lead-in feature 1330, detent snap 1402 of sheath 704 is locked into place in locked groove 1332. As such, detent snap 1402 cannot be easily moved in a distal direction due to a surface with a near perpendicular plane to central axis 1346, shown as detent snap flat 1406 in FIG. 5C.

As housing 702 moves further in a proximal direction toward the skin surface, and as sheath 704 advances toward the distal end of housing 702, detent snaps 1402 shift into the unlocked grooves 1334, and applicator 150 is in an “armed” position, ready for use. When the user further applies force to the proximal end of housing 702, while sheath 704 is pressed against the skin, detent snap 1402 passes over firing detent 1344. This begins a firing sequence due to release of stored energy in the deflected detent snaps 1402, which travel in a proximal direction relative to the skin surface, toward sheath stopping ramp 1338 which is slightly flared outward with respect to central axis 1346 and slows sheath 704 movement during the firing sequence. The next groove encountered by detent snap 1402 after unlocked groove 1334 is final lockout groove 1336 which detent snap 1402 enters at the end of the stroke or pushing sequence performed by the user Final lockout recess 1336 can be a proximally-facing surface that is perpendicular to central axis 1346 which, after detent snap 1402 passes, engages a detent snap flat 1406 and prevents reuse of the device by securely holding sheath 704 in place with respect to housing 702. Insertion hard stop 1322 of housing guide rib 1321 prevents sheath 704 from advancing proximally with respect to housing 702 by engaging sensor electronics carrier travel limiter face 1420.

### Example Embodiment of Applicator Sheath

FIGS. SA and SB are a side view and perspective view, respectively, depicting an example embodiment of sheath 704. In this example embodiment, sheath 704 can stage sensor control device 102 above a user's skin surface prior to application. Sheath 704 can also contain features that help retain a sharp in a position for proper application of a sensor, determine the force required for sensor application, and guide sheath 704 relative to housing 702 during application. Detent snaps 1402 are near a proximal end of sheath 704, described further with respect to FIG. 5C below. Sheath 704 can have a generally cylindrical cross section with a first radius in a proximal section (closer to top of figure) that is shorter than a second radius in a distal section (closer to bottom of figure). Also shown are a plurality of detent clearances 1410, three in the example embodiment. Sheath 704 can include one or more detent clearances 1410, each of which can be a cutout with room for sheath snap lead-in feature 1330 to pass distally into until a distal surface of locking rib 1340 contacts a proximal surface of detent clearance 1410.

Guide rails 1418 are disposed between sensor electronics carrier traveler limiter face 1420 at a proximal end of sheath 704 and a cutout around lock arms 1412. Each guide rail 1418 can be a channel between two ridges where the guide edge 1326 of housing guide rib 1321 can slide distally with respect to sheath 704.

Lock arms 1412 are disposed near a distal end of sheath 704 and can include an attached distal end and a free proximal end, which can include lock arm interface 1416. Lock arms 1412 can lock device carrier 710 to sheath 704 when lock arm interface 1416 of lock arms 1412 engage lock interface 1502 of device carrier 710. Lock arm strengthening ribs 1414 can be disposed near a central location of each lock arm 1412 and can act as a strengthening point for an otherwise weak point of each lock arm 1412 to prevent lock arm 1412 from bending excessively or breaking.

Detent snap stiffening features 1422 can be located along the distal section of detent snaps 1402 and can provide reinforcement to detent snaps 1402. Alignment notch 1424 can be a cutout near the distal end of sheath 704, which provides an opening for user alignment with sheath orientation feature of platform 808. Stiffening ribs 1426 can include buttresses, that are triangularly shaped here, which provide support for detent base 1436. Housing guide rail clearance 1428 can be a cutout for a distal surface of housing guide rib 1321 to slide during use.

FIG. 5C is a close-up perspective view depicting an example embodiment of detent snap 1402 of sheath 704. Detent snap 1402 can include a detent snap bridge 1408 located near or at its proximal end. Detent snap 1402 can also include a detent snap flat 1406 on a distal side of detent snap bridge 1408. An outer surface of detent snap bridge 1408 can include detent snap rounds 1404 which are rounded surfaces that allow for easier movement of detent snap bridge 1408 across interior surfaces of housing 702 such as, for example, locking rib 1340.

FIG. 5D is a side view depicting an example embodiment of sheath 704. Here, alignment notch 1424 can be relatively close to detent clearance 1410. Detent clearance 1410 is in a relatively proximal location on distal portion of sheath 704.

FIG. 5E is an end view depicting an example embodiment of a proximal end of sheath 704. Here, a back wall for guide rails 1446 can provide a channel to slidably couple with housing guide rib 1321 of housing 702. Sheath rotation limiter 1448 can be notches which reduce or prevent rotation of the sheath 704. In a general sense, the embodiments described herein operate by flattening and stretching a skin surface at a predetermined site for sensor insertion. Moreover, the embodiments described herein may also be utilized for other medical applications, such as, e.g., transdermal drug delivery, needle injection, wound closure stitches, device implantation, the application of an adhesive surface to the skin, and other like applications.

By way of background, those of skill the art will appreciate that skin is a highly anisotropic tissue from a biomechanical standpoint and varies largely between individuals. This can affect the degree to which communication between the underlying tissue and the surrounding environment can be performed, e.g., with respect to drug diffusion rates, the ability to penetrate skin with a sharp, or sensor insertion into the body at a sharp-guided insertion site.

### Example Embodiments of Device Carriers

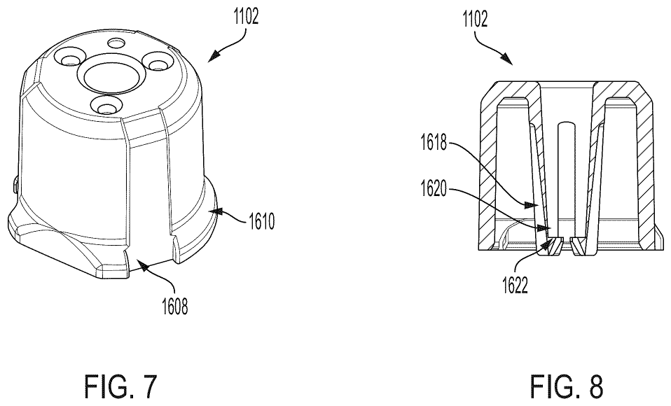

FIG. 6A is a proximal perspective view depicting an example embodiment of device carrier 710 that can retain sensor electronics within applicator 150. It can also retain sharp carrier 1102 with sharp module 2500. In this example embodiment, carrier 710 generally has a hollow round flat cylindrical shape, and can include one or more deflectable sharp carrier lock arms 1524 (e.g., three) extending proximally from a proximal surface surrounding a centrally located spring alignment ridge 1516 for maintaining alignment of spring 1104. Each lock arm 1524 has a detent or retention feature 1526 located at or near its proximal end. Shock lock 1534 can be a tab located on an outer circumference of device carrier 710 extending outward and can lock device carrier 710 for added safety prior to firing. Rotation limiter 1506 can be a proximally extending relatively short protrusion on a proximal surface of device carrier 710 which limits rotation of carrier 710. Sharp carrier lock arms 1524 can interface with sharp carrier 1102 as described with reference to FIGS. 7 and 8 below.

FIG. 6B is a distal perspective view of device carrier 710. Here, one or more sensor electronics retention spring arms 1518 (e.g., three) are normally biased towards the position shown and include a detent 1519 that can pass over the distal surface of electronics housing 706 of device 102 when housed within recess or cavity 1521. In certain embodiments, after sensor control device 102 has been adhered to the skin with applicator 150, the user pulls applicator 150 in a proximal direction, i.e., away from the skin. The adhesive force retains sensor control device 102 on the skin and overcomes the lateral force applied by spring arms 1518. As a result, spring arms 1518 deflect radially outwardly and disengage detents 1519 from sensor control device 102 thereby releasing sensor control device 102 from applicator 150.

### Example Embodiments of Sharp Carriers

FIGS. 7 and 8 are a proximal perspective view and a side cross-sectional view, respectively, depicting an example embodiment of sharp carrier 1102. Sharp carrier 1102 can grasp and retain sharp module 2500 within applicator 150. Near a distal end of sharp carrier 1102 can be anti-rotation slots 1608 which prevent sharp carrier 1102 from rotating when located within a central area of sharp carrier lock arms 1524 (as shown in FIG. 6A). Anti-rotation slots 1608 can be located between sections of sharp carrier base chamfer 1610, which can ensure full retraction of sharp carrier 1102 through sheath 704 upon retraction of sharp carrier 1102 at the end of the deployment procedure.

As shown in FIG. 8, sharp retention arms 1618 can be located in an interior of sharp carrier 1102 about a central axis and can include a sharp retention clip 1620 at a distal end of each arm 1618.

### Example Embodiments of Sensor Structures and Features Related Thereto

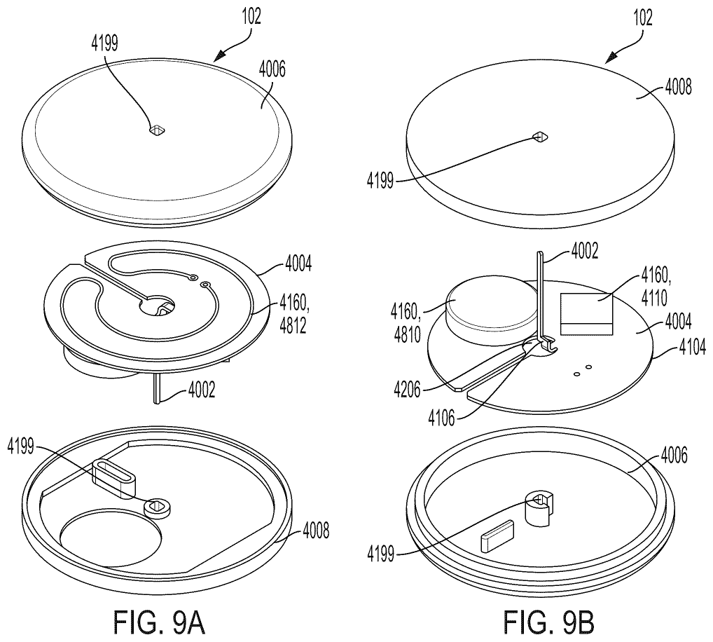

FIGS. 9A and 9B are top and bottom exploded views, respectively, depicting an example embodiment of a sensor control device 102 configured to house an analyte sensor 4104 integrated with sensor electronics 4160. According to an aspect of the embodiments, the analyte sensor 4104 comprises an in vivo portion 4002, an ex vivo portion 4004, and a neck 4106 which interconnects the ex vivo portion 4104 and the in vivo portion 4002 and allows folding of the analyte sensor 4104, for example ninety degrees. The in vivo portion 4002 can have a first surface and a second surface, and can be configured to be positioned in contact with an interstitial fluid of a user and to generate signals associated with a measured analyte level. The in vivo portion 4002 can be the portion of the analyte sensor 4104 that resides under the user's skin after insertion. The ex vivo portion 4004 can comprise a plurality of sensor electronics 4160 mounted thereon. In some embodiments, an integral, monolithic sensor having an in vivo portion 4002 comprising a substrate with one or more electrodes printed thereon and an ex vivo portion 4004 having the substrate with sensor electronics 4160 mounted thereon can be formed.

According to an aspect of the embodiments, an integral, monolithic sensor can be advantageous in reducing the number of required components, thereby reducing the overall size of the sensor control device, reducing manufacturing complexity and cost, and potentially increasing user access to these devices. By reducing the size, comfort and convenience to the user can be improved. According to embodiments disclosed here, mounting the sensor electronics 4160 on the substrate of the analyte sensor on the ex vivo portion 4004 to comprise a single component eliminates the need to electrically couple the analyte sensor 4104 with a circuit board using an electrical connector, thereby enabling reduction of the size of the sensor control device 102 and increasing the reliability of the connection between the electronic components and the circuit board. By eliminating the need for an electrical connector, and by shrinking the size of the overall sensor control device 102, manufacturing and purchase costs can be reduced. Indeed, in order to measure multiple analytes, one working electrode for each analyte is required. As a result, an analyte sensor 4104 configured to measure multiple analytes includes a corresponding number of multiple working electrodes. The greater the number of electrodes in an analyte sensor 4104, the larger the connector needs to be, further exacerbating the problem for multiple analyte sensors. A reduced size and lower associated cost of manufacture is advantageous because the sensor control device 102 is single use with a limited lifespan, thereby requiring frequent replacement.

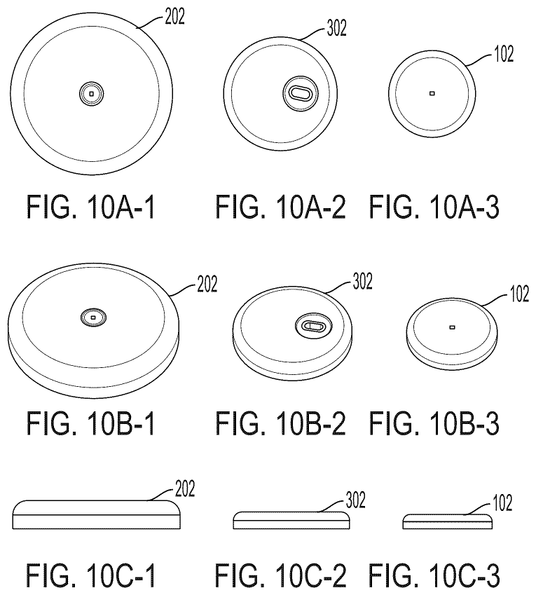

FIGS. 10A-1 and 10A-2 are top perspective views of previous embodiments of sensor control devices 202, 302, respectively. FIG. 10A-3 is a top perspective view of the sensor control device 102, illustrating the overall reduced cross-sectional area of the sensor control device 102 when compared to previous embodiments. FIGS. 10B-1 and 10B-2 are additional perspective views of the sensor control devices 202, 302 depicted in FIGS. 10A-1 and 10A-2, and FIG. 10C-1 is side perspective view of the sensor control device 102 described herein, further illustrating the size discrepancy between the embodiment described herein and previously described sensor control device embodiments. Further, FIGS. 10C-1 and 10C-2 are side views of the sensor control devices 202, 302 depicted in FIGS. 10A-1 and 10A-2, and FIG. 10C-3 is a side view of the sensor control device 102 described herein, illustrating the reduced height of the sensor control device when compared with previous embodiments of the sensor control device.

In some embodiments, and with reference to FIGS. 9A and 9B, the ex vivo portion 4004 can be any suitable shape including but not limited to circular or semi-circular. Those of skill in the art will recognize various other suitable shapes that can be utilized for the ex vivo portion 4004 of the analyte sensor 4104 without departing from the scope of the disclosure. In some embodiments, and as depicted in FIGS. 9A-9B, the in vivo portion 4002 can be centrally or substantially centrally located with respect to the ex vivo portion 4004. In some embodiments, and as best shown in FIG. 9B, the in vivo portion 4002 can extend through a space or aperture 4206 in the ex vivo portion 4004. As will be appreciated in the art, the in vivo portion 4002 can also be located offset from the center of the ex vivo portion 4004, or positioned in any other suitable location.

According to an aspect of the embodiments, the sensor electronics 4160 can be positioned at any suitable location on the ex vivo portion 4004, and on either or both surfaces of the ex vivo portion 4004. For example, in some embodiments, and as best depicted in FIG. 9B, a battery 4810 and an ASIC 4410 can be positioned on a first surface of the ex vivo portion 4004, and an antenna 4812 can be positioned on a second surface of the ex vivo portion 4004 (see FIG. 9A). In some embodiments, the antenna 4812 can be embedded within a surface of the ex vivo portion 4004 (see, e.g., FIG. 9A). For example, and as best shown in FIG. 9A, the antenna 4812 can be provided in a looped or threaded manner such that a conductive trace is provided on the second surface (or first surface, in some embodiments) of the ex vivo portion 4004 along most or all of a perimeter thereof, wherein a portion of the conductive trace wraps into an inner perimeter of the ex vivo portion 4004 so as to form at least two loops.

According to another aspect of the embodiments, the analyte sensor 4104 can include an in vivo portion 4002 having a substrate, at least one working electrode, and a reference electrode configured such that the electrodes are printed on the substrate. Each of the at least one working electrodes can be configured to measure an analyte of interest (such as, without limitation, glucose, ketone, lactate, etc.).

In some embodiments, the ex vivo portion 4004 of the analyte sensor 4104 can comprise a plurality of sensor electronics 4160. The plurality of sensor electronics 4160 can be communicatively coupled to the at least one working electrode and reference electrode, and can be configured to receive signals associated with the measured analyte level generated by the electrodes. Specifically, the ex vivo portion 4004 can include sensor electronics 4160 mounted thereon for receiving the analyte measurement signals generated by the analyte sensor 4104 in the in vivo portion 4002. In some embodiments, the sensor electronics 4160 can be mounted on the substrate of the ex vivo portion 4004 or the ex vivo portion 4004 of the analyte sensor 4104. As a result, a separate printed circuit board is not needed to mount electronic components.

Similar to the sensor electronics 160 embodiment illustrated in FIGS. 2B and 2C, the sensor electronics 4160 depicted in FIGS. 9A-9B can have a majority of the processing capability for rendering end-result data suitable for display to the user. The sensor electronics 4160 can include, for example without limitation, one or more processors, resistors, transistors, capacitors, inductors, diodes, switches, a single semiconductor chip (e.g., an ASIC 4110), a power source (e.g., one or more batteries 4810), and/or one or more antennae 4812. Specifically, in some embodiments, the antenna 4812 can be an NFC antenna 4812 that utilizes an NFC communication protocol, thereby reducing cost and size of the sensor control device 102 by eliminating the need for Bluetooth. However, those of skill in the art will recognize that various other communication protocols can be utilized without departing from the scope of the disclosure.

Further, and as shown in FIGS. 9B, the sensor electronics 4160 can include a power source, such as one or more batteries 4810. As depicted in FIG. 9B, the batteries or battery 4810 can include, for example without limitation, a coin battery. Those of skill in the art will appreciate that other types of batteries can be utilized, for example, one or more fed batteries or printed batteries, without departing from the scope of this disclosure.

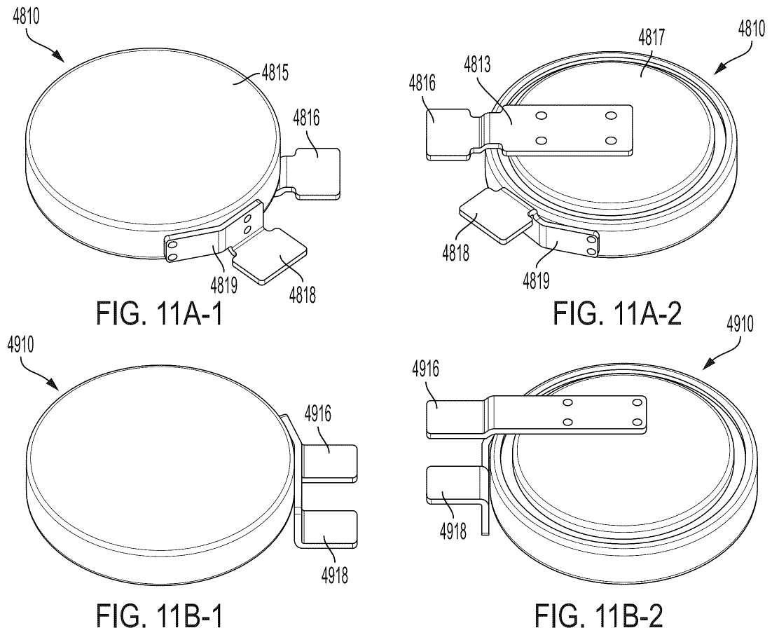

Turning to FIGS. 11A-1 and 11A-2, top and bottom perspective views of an exemplar embodiment of the battery 4810 are shown, wherein the battery 4810 can be utilized with sensor control device 102 comprising the analyte sensor 4104 with integrated sensor electronics 4160. Specifically, FIG. 11A-1 depicts a negatively charged surface 4815 of the battery 4810 and FIG. 11A-2 depicts a positively charged surface 4817 of the battery 4810. Specifically, and as shown in FIGS. 11A-1 and 11A-2, the battery 4810 can be a coin battery 4810 comprising one or more tabs 4816, 4818. More specifically, the battery 4810 can comprise one or more side tabs 4816, 4818 extending from a periphery of the battery 4810. As best depicted in FIG. 11A-2, the battery 4810 can include a first tab 4816 and a second tab 4818, wherein the first tab 4816 comprises and extends from a first tab base 4813 welded on the positively charged surface 4817 of the battery 4810. In this regard, the first tab base 4813 is adhered to the positively charged surface 4817 of the battery and the first tab 4816 is extending from the periphery of the battery 4810 so as to additionally reduce height. Further, in some embodiments, and as shown in FIGS. 11A-1 and 11A-2, the second tab 4818 can comprise and extend from a second tab base 4819 welded on an edge portion 4811 of the battery 4810.

Still referring to FIGS. 11A-1 and 11A-2, the first tab 4816 and second tab 4818 can be oriented radially relative to one another. In some embodiments, the first tab 4816 can be oriented 45-degrees from the second tab 4818. As illustrated in FIGS. 11A-1 and 11A-2, the first tab 4816 and second tab 4818 can be oriented such that they are coplanar. In some embodiments, the first tab 4816 and second tab 4818 can be oriented such that they are non-coplanar. FIGS. 11B-1 and 11B-2 depict an additional exemplar embodiment of a battery 4910 to be utilized with the sensor control device 102. Specifically, the battery 4910 shown in FIGS. 11B-1 and 11B-2 is similar to the battery 4810 depicted in FIGS. 11A-1 and 11A-2, except that a first tab 4916 and a second tab 4918 are oriented such that they are parallel and/or symmetric.

Additional exemplar embodiments of analyte sensors with integrated sensor electronics are shown in FIGS. 12A-1 to 12B-4. Specifically, with reference to FIGS. 12A-1 to 12A-4, an analyte sensor 4134 with integrated sensor electronics 4160 is shown. Analyte sensor 4134 with integrated sensor electronics 4160 is similar to the analyte sensor 4104 with integrated sensor electronics 4160 depicted in FIGS. 9A and 9B, except that at least a portion of the ex vivo portion 4304 of the analyte sensor 4134 is folded (e.g., in half) such that the size of the sensor control device 102 is further reduced. In some embodiments, the substrate on the ex vivo portion 4304 or, the analyte sensor 4134, in general, can be made from or composed of any flexible non-electrically-conductive polymer. For example, the substrate on the ex vivo portion 4304 or, the analyte sensor 4134, can be made of polyamide, polyester, polyethylene terephthalate (PET), or a substrate of the like which allows the ex vivo portion 4304 to be flexible. Due to the flexible nature of the substrate, and because the sensor electronics 4160 are directly mounted onto the substrate without the need for a connector, the substrate is able to be folded, as shown in FIGS. 12A-3 and 12A-4. In some embodiments, the sensor electronics 4160 are mounted to the ex vivo portion using photonics soldering.

Specifically, the ex vivo portion 4304 (or, the analyte sensor 4134, in general) and the sensor electronics 4160 can be formed from a one-piece substrate More specifically, the substrate can be cut-out, layered out, stamped out, or molded. The substrate can be formed through a die-cutting process, laser-cutting process, or ultrasonic cutting process. The substrate can also be fabricated through a 3-D printing process. If the substrate is formed through a molding process, the substrate can be injection, compression, or blow-molded to create three dimensional features.

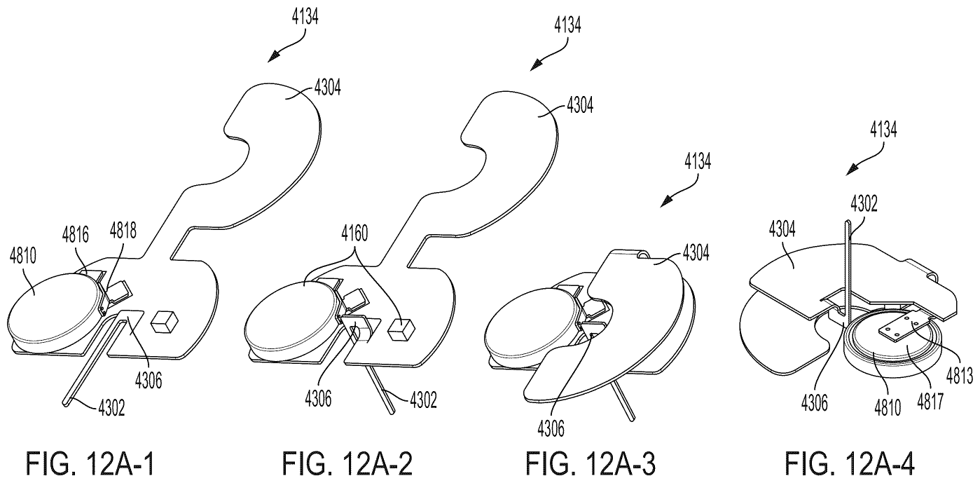

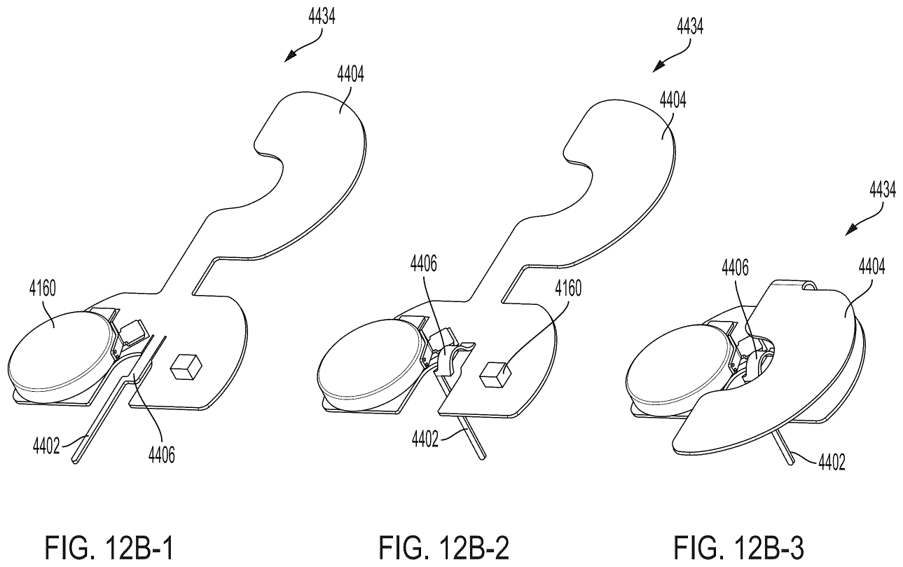

In the embodiment shown in FIGS. 12A-1 to 12A-4, the neck 4306 of the analyte sensor 4134 comprises a single fold. In this embodiment, though not depicted, the antenna 4812 is also configured to be folded. Further, the in vivo portion 4302 of the analyte sensor 4134 is configured to be exposed such that it can be dipped in a membrane material to form a membrane. Specifically, the membrane on the in vivo portion 4302 can cover an active analyte sensing element of the analyte sensor 4134 or regulate analyte influx. In the embodiment depicted in FIGS. 12A-1 to 12A-4, the sensor electronics 4160 includes a battery 4810 similar to the battery embodiment depicted in FIGS. 11A-1 and 11A-2, wherein the first tab 4816 is oriented radially from the second tab 4818 and is coplanar therewith. Similar to the battery 4810 embodiment illustrated in FIGS. 11A-1 and 11A-2, the first tab 4816 is welded on the positively charged surface 4817 of the battery 4810 by the first tab base 4813 (shown in FIG. 12A-4) so as to further reduce height.

Specifically, FIG. 12A-1 depicts a top perspective view of the analyte sensor 4134 integrated with sensor electronics 4160, wherein the ex vivo portion 4304 is in an unfolded position and the analyte sensor 4134 is not bent at the neck 4306 such that the in vivo portion 4302 and the ex vivo portion 4304 form a substantially planar or planar surface. FIG. 12A-2 is a top perspective view of the analyte sensor 4134 integrated with sensor electronics 4160, wherein the ex vivo portion 4304 is in an unfolded position and the neck 4306 of the analyte sensor 4134 is bent with a single fold such that the in vivo portion 4302 is at an angle (e.g., about ninety degrees) relative to the ex vivo portion 4304. FIGS. 12A-3 and 12A-4 are top and bottom perspective views, respectively, of the analyte sensor 4134 integrated with sensor electronics 4160, wherein the ex vivo portion 4304 is in a folded position and the analyte sensor 4134 is bent with a single fold such that the in vivo portion 4302 is at an angle (e.g., about ninety degrees) relative to the ex vivo portion 4304. In some embodiments, and as best shown in FIGS. 12A-1 to 12A-3, the battery 4810 can be welded onto the substrate of the ex vivo portion 4304 by the first tab 4816 and second tab 4818, but the battery 4810 itself can be offset from the ex vivo portion 4304 or its substrate so as to further reduce height. In other words, the battery 4810 is mounted onto the ex vivo portion 4304 by the first tab 4816 and second tab 4818, but the ex vivo portion 4304 is not disposed underneath a surface of the battery 4810 (e.g., the ex vivo portion 4304 is not disposed underneath the positively charged surface 4817 or negatively charged surface 4815 of the battery 4810, such that at least a portion of the battery 4810 is hanging from a periphery or portion of the ex vivo portion 4304, as best shown in FIG. 12A-4).

FIGS. 12B-1 to 12B-3 depict top perspective views of an additional exemplar embodiment of an analyte sensor 4434 integrated with sensor electronics 4160. The analyte sensor 4434 embodiment depicted in FIGS. 12B-1 to 12B-3 is similar to the analyte sensor 4134 embodiment shown in FIGS. 12A-1 to 12A-4, except that the analyte sensor 4234 comprises a neck 4406 having a double fold. Specifically, FIG. 12B-1 depicts a top perspective view of the analyte sensor 4434 integrated with sensor electronics 4160, wherein the ex vivo portion 4404 is in an unfolded position and the analyte sensor 4434 is not bent at the neck 4406 such that the in vivo portion 4402 and the ex vivo portion 4404 form a substantially planar or planar surface. FIG. 12B-2 is a top perspective view of the analyte sensor 4434 integrated with sensor electronics 4160, wherein the ex vivo portion 4404 is in an unfolded position and the neck 4406 of the analyte sensor 4434 is bent with the double fold such that the in vivo portion 4402 is at an angle (e.g., about ninety degrees) relative to the ex vivo portion 4404. FIG. 12B-3 depicts the analyte sensor 4434 integrated with sensor electronics 4160, wherein the ex vivo portion 4404 is in a folded position and the analyte sensor 4434 is bent with the double fold neck 4406 such that the in vivo portion 4402 is at an angle (e.g., about ninety degrees) relative to the ex vivo portion 4404.

Those of skill in the art will appreciate that other battery embodiments (e.g., battery 4910) can be utilized with analyte sensor 4104, 4134, or 4434 without departing from the scope of the disclosure.

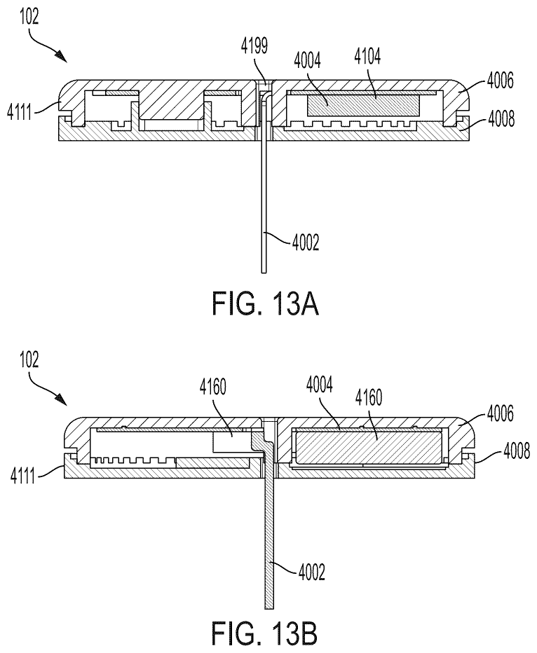

As illustrated in FIGS. 9A-9B and 13A-13B, the enclosure of the sensor control device 102 can include elements welded (or snap-fit/adhered) together. In some embodiments, the enclosure of the sensor control device 102 can include an upper portion 4006 and mount portion 4008 which form a unitary piece or housing 4111. Further, in some embodiments, an aperture can extend through the upper portion and mount portion. In some embodiments, the in vivo portion 4002 is configured to extend through at least part of the aperture 4199 in the mount portion 4008. The upper portion 4006 and mount portion 4008 are configured to sealably enclose and protect the sensor electronics 4160. FIGS. 13A-13B are side cutaway views further depicting the sensor control device 102 comprising the analyte sensor 4104 and integrated sensor electronics 4160 enclosed within the upper portion 4006 and mount portion 4008. As best depicted in FIGS. 13A and 13B, the upper portion 4006 and mount portion 4008 interface to form the housing 4111 with a hollow interior.

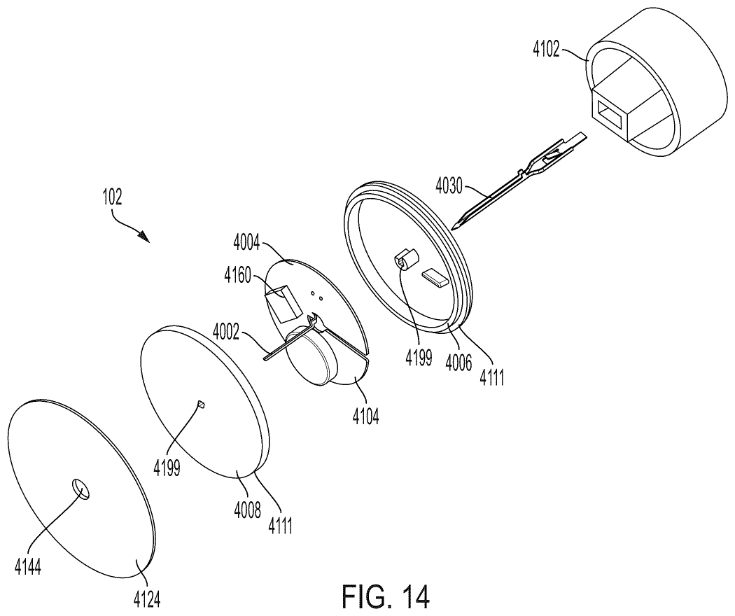

FIG. 14 is an exploded view depicting the sensor control device 102 comprising the housing 4111. As illustrated in FIG. 14, the sensor control device can further comprise an adhesive patch 4124. The adhesive patch 4124 is arranged on the base surface of the housing which, in some embodiments, is on a distal surface of the mount portion of the housing 4111. The adhesive patch 4124 comprises an adhesive on the distal surface. The adhesive patch 3124 is provided for attaching the sensor control device 102 to the user's skin. According to an aspect of the embodiments, the adhesive patch 4124 comprises a hole 4144 corresponding and aligned with the aperture 4199 which extends through the upper portion 4006 and mount portion 4008 such that the in vivo portion 4002 can pass therethrough and be implanted under the user's skin.

Specifically, and with reference to FIG. 13A to 14, at least a portion of the analyte sensor 4104 is configured to protrude below the sensor control device 102. In particular, the in vivo portion extends distally from the mount portion 4008 of the sensor control device 102 and through the hole 4144 of the adhesive patch 4124 (see FIG. 14). This means that as the sensor control device is placed onto the skin, at least a portion (e.g. the in vivo portion) of the analyte sensor is inserted into the skin. For example, this allows the analyte sensor 4104 to be in contact with interstitial fluid of the user. Another portion of the sensor 4104 (e.g., the ex vivo portion 4004) is integrated with the sensor electronics 4160 within the housing 4111 of the sensor control device 102. In exemplar embodiments, the in vivo portion 4002 is arranged longitudinally (e.g. vertically) while the ex vivo portion 4004 is at an angle, such as perpendicular (e.g. horizontal) within the housing of the sensor control device 102.

According to exemplar embodiments, and with reference to FIG. 14, a sharp 4030 and sharp carrier 4102 can be provided to facilitate insertion of the analyte sensor's in vivo portion 4002. Specifically, and as best shown in FIG. 14, at least a portion of the sharp 4030 is configured to couple to the housing 4111 of the sensor control device 102.

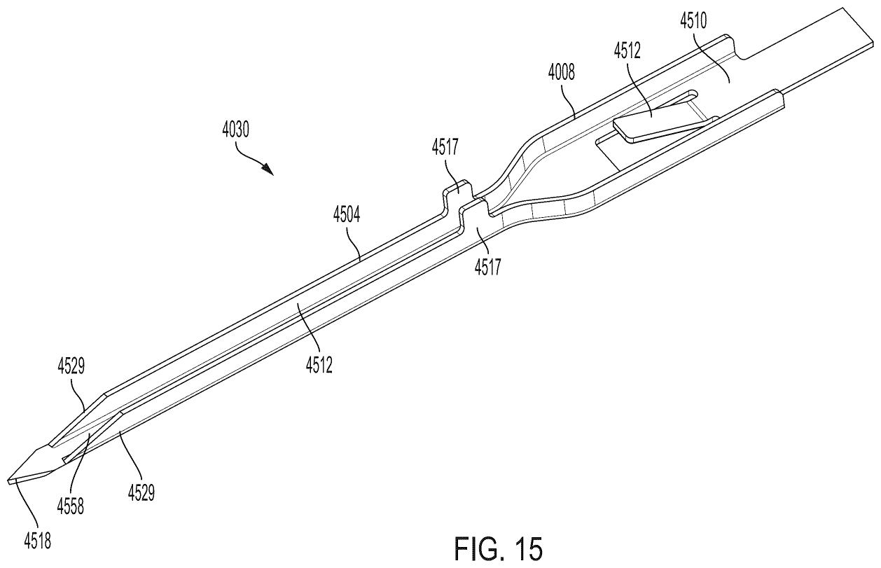

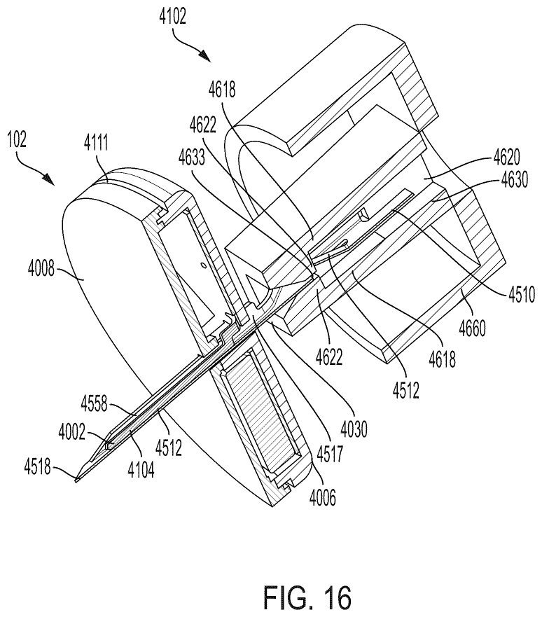

FIG. 15 is a perspective view depicting an example embodiment of a sharp 4030, and FIG. 16 is a cutaway view depicting an example embodiment of a sharp carrier 4102 (comprising the sharp 4030). With particular reference to FIG. 15, sharp 4030 can include an upper portion 4510 and a lower portion 4512. In some embodiments, the upper portion 4510 is provided with a larger surface area than the lower portion 4512, and comprises a cantilever arm 4515 that is configured to engage with or snap into the sharp carrier 4102. In some embodiments, a sharp window is provided on the upper portion 4515 and is configured to engage or snap with the sharp carrier 4102. Further, a pair of sharp tabs 4517 are provided on the sharp 4030. Specifically, the sharp tabs 4517 are disposed between the upper portion 4510 and the lower portion 4512 of the sharp 4030, and are configured to mate with the upper portion 4006 of the sensor control device's housing 4111 (see, e.g., FIG. 14). In this regard, the upper portion 4510 of the sharp 4030 is configured to be positioned proximal relative to the upper portion 4006 of the sensor control device 102.

Specifically, and with particular reference to FIGS. 15 and 16, the sharp 4030 can be configured with a distal tip 4518 which can penetrate the skin while carrying the in vivo portion 4002 of the analyte sensor 4104 (see FIG. 16) in a hollow or recess of sharp shaft 4504 to put the active surface of the in vivo portion 4002 in contact with bodily fluid. More specifically, and as best shown in FIG. 15, the sharp 4030 can comprise a sensor channel 4558 configured to receive at least a portion of the analyte sensor 4104 (e.g., the in vivo portion 4002). In this manner, the in vivo portion 4002 of the analyte sensor 4104 (not shown in FIG. 15) has a width sized to fit within the sensor channel 4558. This allows the sharp 4030 to be used to insert the analyte sensor 4104. According to some embodiments, and as depicted in FIG. 15, one or more sidewalls 4529 that form the sensor channel 4558 are disposed along the sharp shaft 4504 at a predetermined distance. In other words, according to some embodiments, the sensor channel 4558 is in a spaced relation to the distal tip 4518. In this regard, the distal tip 4518 has a reduced cross-sectional footprint relative to, for example, a sharp having a distal tip with the sensor channel directly adjacent thereto.

According to one aspect of the embodiments, and as best shown in FIG. 16, the sharp 4030 or at least a portion thereof extends through the sensor control device 102. In particular, the lower portion 4512 of the sharp 4030 is configured to protrude downwardly from the mount portion 4008 of the sensor control device 102. Thus, the sensor control device 102 is configured to receive the sharp 4030 therethrough. The aperture 4199 and hole 4144 (not depicted in FIG. 16) are aligned with the in vivo portion 4002 of the analyte sensor 4104 so that the sharp 4030 can be arranged adjacent the analyte sensor 4104. The sharp 4030 can thus extend through the upper portion 4006 and the mount portion 4008, or the housing 4111. The sharp 4030 can further pass through the hole 4144 in the adhesive patch (not shown in FIG. 16) so as to allow the analyte sensor 4104 to make contact with interstitial fluid of the user. Further, according to some embodiments, the sharp 4030 can include a U-shaped or V-shaped geometry.

Referring to FIGS. 15 and 16, the cantilever arm 4512 of the sharp's upper portion 4510 can provide a surface for a sharp carrier 4102 to retain and grasp. Specifically, the sharp's upper portion 4510 can be coupled to a distal end of the sharp carrier 4102 (as shown in FIG. 16). More specifically, sharp retention walls 4618 can be located in a cavity 4620 of an inner portion 4550 of the sharp carrier 4102 about a central axis and can include one or more sharp retention shoulders 4622 at a distal end of each wall 4618. Sharp retention shoulders 4622 can have a proximal surface which can be nearly perpendicular to the central axis and can abut the upper portion 4510 of the sharp 4020. Specifically, one or more sharp retention shoulders can abut the cantilever arm of the sharp. More specifically, one or more sharp retention shoulders 4622 can abut a distal end of the cantilever arm 4618. In this manner, the sharp 4030 is securely retained by the sharp carrier 4102 and is protected from dropping out.

Still with reference to FIGS. 16, in some embodiments, the sharp carrier 4102 comprises a smaller diameter inner portion 4630 which extends downwardly from a larger diameter, hollow outer portion 4660. In some embodiments, the outer portion 4660 of the sharp carrier 4102 is cylindrical or substantially cylindrical in shape, and the inner portion 4630 of the sharp carrier 4102 is rectangular or substantially rectangular in shape. Those of skill in the art will recognize that other shapes can be utilized for the outer portion 4660 and inner portion 4630 without departing from the scope of the disclosure.

Further, in some embodiments, the inner portion 4630 of the sharp carrier 4102 comprises a sharp channel 4633 on a distal surface thereof so as to allow the upper portion 4510 of the sharp 4030 to extend therethrough and into the cavity 4620. In some embodiment, the sharp channel 4633 extends into and forms the cavity 4620. Further, in some embodiments, the cavity 4620 extends from a distal surface of the inner portion 4630 to a proximal surface of the inner portion 4630. In some embodiments, an opening on a top surface of the outer portion 4660 is longitudinally aligned with the proximal surface of the inner portion 4630 such that the cavity is directly adjacent to and distal relative to the opening.

In some embodiments, a width of the sharp channel 4633 is smaller than a width of the cavity 4620. In some embodiments, when the sharp 4030 is securely retained by the sharp carrier 4102, the portion of the sharp 4030 that is distal relative to the cantilever arm 4512, and at least a portion of the sharp 4030 that is proximal relative to the sharp tabs 4517 can be oriented so as to interface with the sharp channel 4633. Further, and according to an aspect of the embodiments, the sharp channel 4633 of the sharp carrier 4102 is configured such that it axially aligns with the aperture 4199 of the sensor control device's housing 4111 and the hole 4144 of the adhesive patch 4124 (not shown in FIG. 16) In this manner, the sharp 4030 can extend through the sharp carrier 4102 and sensor control device 102 to facilitate analyte sensor insertion.

According to an aspect of the embodiments, the sensor control devices 102 described herein comprising the analyte sensor 4104 integrated with sensor electronics 4160 can undergo low energy terminal e-beam sterilization. Further, in some embodiments, a spray can be utilized to make the sensor control device 102 waterproof. A waterproof sensor control device 102 can be particularly useful for user's engaging in outdoor and/or athletic activities. To achieve the desired waterproof state of the sensor control device 102, a spray can be utilized which comprises silicone, polyurethane, a wax-based formula, or the like. In some embodiments, the spray can comprise an adhesive property.

According to another aspect of the embodiments, though not illustrated, the sensor control devices 102 described herein can be colored customized so as to match a skin tone of the user. Customized coloring and unique designs of the sensor control device 102 can be provided by utilizing 3D-printing for the housing 4111. In some embodiments, a sticker can be applied to the housing of the sensor control device 102, wherein the sticker comprises the desired color or design.

According to yet another aspect of the embodiments, though not shown, the sensor control devices 102 described herein can comprise a display for alarms or ringlike to show color. Further, the sensor control device 102 can be configured to provide vibratory or tactile feedback. Additionally, in some embodiments, the sensor control device 102 can provide auditory feedback to indicate upward or downward movements related to the insertion process so as to assist individuals who may be visually impaired or blind. In some embodiments, the sensor control device 102 can be configured to connect to WiFi and/or the television. In some embodiments, the sensor control device 102 can be connected to and in communication with other devices, e.g., smart watches, televisions, wearables, smart phones with operating systems, “Apple Homekit,” reader devices, and the like. Further, the sensor control device 102 can be linked to hospital systems such that patients can be monitored by a healthcare provider.

Furthermore, it will be understood by those of skill in the art that the sensor control device embodiments described herein can similarly be used with any of the analyte sensors described herein, including embodiments of features related thereto, such as sensor electronics.

### Exemplary Firing Mechanism of Applicators

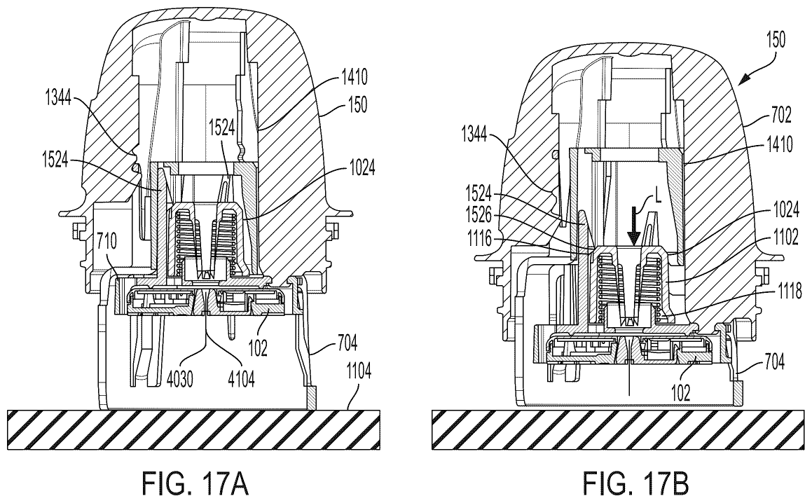

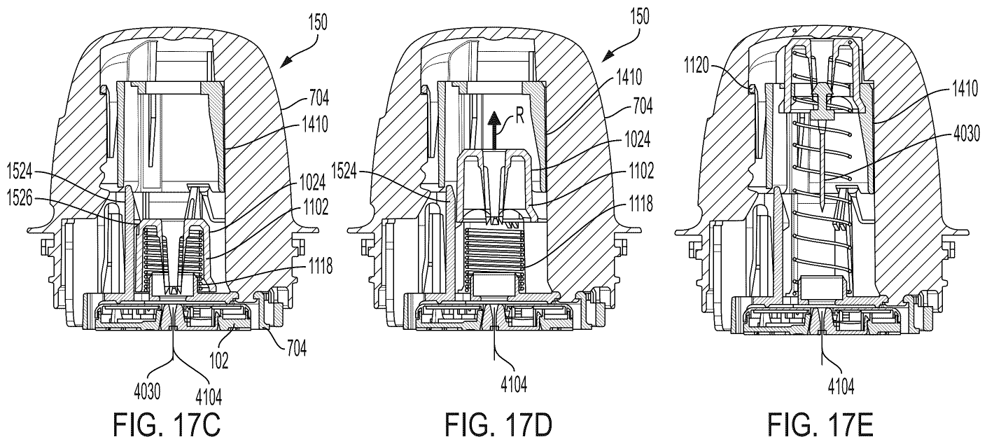

FIGS. 17A-17E illustrate example details of embodiments of the internal device mechanics of “firing” the applicator 150 to apply sensor control device 102 to a user and including retracting sharp 4030 safely back into used applicator 150. All together, these drawings represent an example sequence of driving sharp 4030 (supporting an analyte sensor 4104 coupled to sensor control device 102) into the skin of a user, withdrawing the sharp 4030 while leaving the analyte sensor 4104 behind in operative contact with interstitial fluid of the user, and adhering the sensor control device 102 to the skin of the user with an adhesive patch 4124. Modification of such activity for use with the alternative applicator assembly embodiments and components can be appreciated in reference to the same by those with skill in the art. Moreover, applicator 150 may be a sensor applicator having one-piece architecture or a two-piece architecture as disclosed herein.

Turning now to FIG. 17A, a sensor 4104 is supported within sharp 4030, just above the skin 1104 of the user. The sheath 704 is held by detents 1344 within the applicator 150 such that appropriate downward force along the longitudinal axis of the applicator 150 will cause the resistance provided by the detents 1344 to be overcome so that sharp 4030 and sensor control device 102 can translate along the longitudinal axis into (and onto) skin 1104 of the user. In addition, deflectable sharp carrier lock arms 1524 of device carrier 710 engage the sharp retraction assembly 1024 to maintain the sharp 4030 in a position relative to the sensor control device 102.

In FIG. 17B, user force is applied to overcome or override detents 1344 and sheath 704 collapses into housing 702 driving the sensor control device 102 (with associated parts) to translate down as indicated by the arrow L along the longitudinal axis. An inner diameter of the sheath 704 constrains the position of deflectable sharp carrier lock arms 1524 through the full stroke of the sensor/sharp insertion process. The retention of the retention features 1526 of deflectable sharp carrier lock arms 1524 against complimentary faces 1116 of the sharp carrier 1102 (in some embodiments, an exterior surface of the sharp carrier 4102) maintains the position of the members with return spring 1118 fully energized.

In FIG. 17C, sensor 4104 and sharp 4030 have reached full insertion depth. In so doing, the deflectable sharp carrier lock arms 1524 clear the inner diameter of sheath 704. Then, the compressed force of the coil return spring 1118 drives retention features 1526 radially outward, releasing force to drive the sharp carrier 1102 or 4102 of the sharp retraction assembly 1024 to pull the (slotted or otherwise configured) sharp 4030 out of the user and off of the sensor 4104 as indicated by the arrow R in FIG. 17D.

With the sharp 4030 fully retracted as shown in FIG. 17E, the sheath 704 comprises a final locking feature 1120. Subsequently, the spent applicator assembly 150 is removed from the insertion site, leaving behind the sensor control device 102, and with the sharp 4030 secured safely inside the applicator assembly 150. The spent applicator assembly 150 is now ready for disposal.

Operation of the applicator 150 when applying the sensor control device 102 is designed to provide the user with a sensation that both the insertion and retraction of the sharp 4030 is performed automatically by the internal mechanisms of the applicator 150. In other words, the present invention avoids the user experiencing the sensation that he is manually driving the sharp 4030 into bis skin. Thus, once the user applies sufficient force to overcome the resistance from the detent features of the applicator 150, the resulting actions of the applicator 150 are perceived to be an automated response to the applicator being “triggered.” The user does not perceive that he is supplying additional force to drive the sharp 4030 to pierce his skin despite that all the driving force is provided by the user and no additional biasing/driving means are used to insert the sharp 4030. As detailed above in FIG. 17C, the retraction of the sharp 4030 is automated by the coil return spring 1118 of the applicator 150.

### Exemplary Embodiment of a Flexible Sensor Control Device and Features Related Thereto

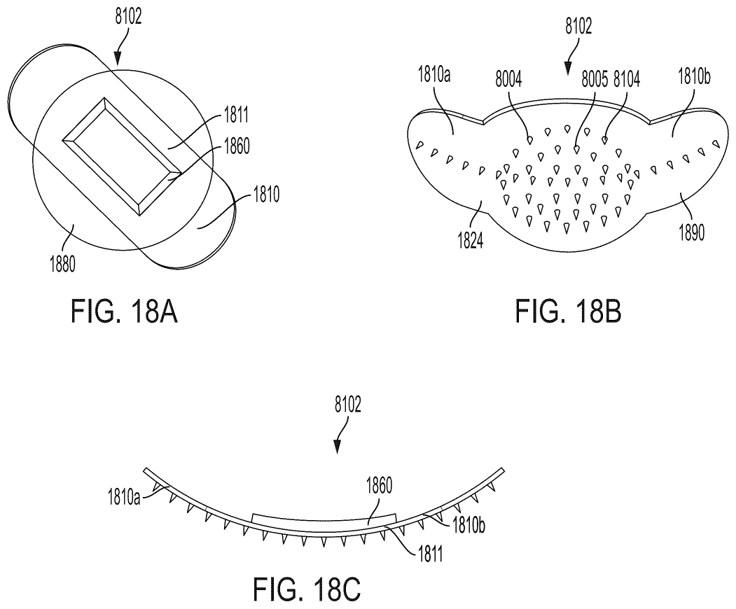

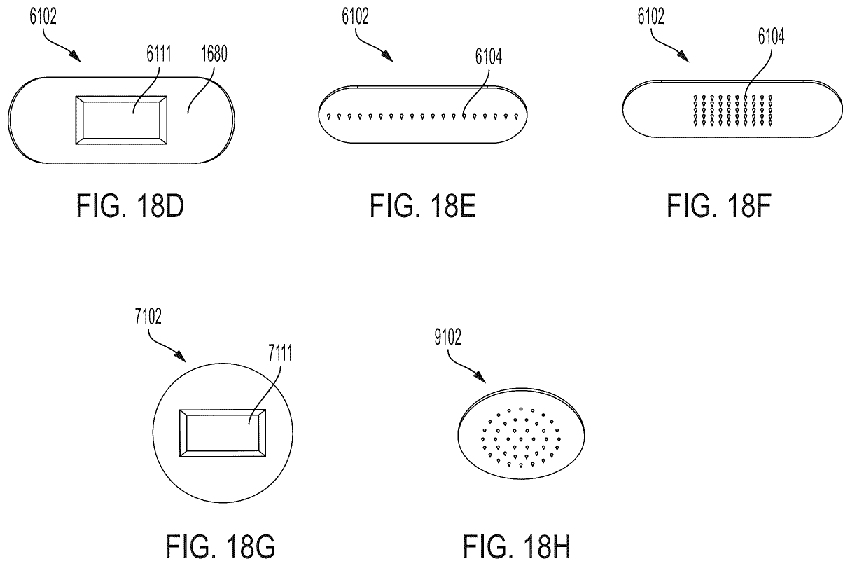

FIGS. 18A and 18B are top perspective and bottom perspective views, respectively, of an example sensor control device 8102, according to one or more embodiments of the present disclosure. The sensor control device can generally be “band-aid” shaped, or comprise a flexible strip with one or more rounded edges. According to an aspect of the embodiments, the sensor control device 8102 is partially or entirely flexible. The flexibility of the sensor control device 8102 allows the sensor control device 8102 to contour to a user's body. Further, in some embodiments, and with reference to FIGS. 18A-18C, the sensor control device 8102 comprises one or more flexible portions 1810 and a structurally rigid portion 1811. In some exemplar embodiments, the structurally rigid portion 1811 can be disposed between the one or more flexible portions 1810. In some embodiments, the structurally rigid portion 1811 is integrated into the shape of the sensor control device 8102. In some embodiments, the flexible portion(s) 1810 can form a base for the structurally rigid portion 1811 to be disposed thereon. In other embodiments, the structurally rigid portion 1811 has a circular cross-section and is generally circular, oval, or ovoid in shape. Those of skill in the art will recognize that other sensor control device 8102 and structurally rigid portion 1811 shapes and cross-sections can be utilized without departing from the scope of the disclosure.

In some embodiments, the structurally rigid portion 1811 forms the electronics housing 1860 and houses the sensor electronics 1860 (not illustrated) therein. In this regard, the stiffness of the structurally rigid portion 1811 maintains rigidity over the sensor electronics 1860 (not illustrated) so as to stabilize and hold the sensor electronics 1860 in place. FIG. 18C is a side perspective view of sensor control device 8102, illustrating a first flexible portion 1810a, a second flexible portion 1810b, and the structurally rigid portion 1811 which houses the sensor electronics 1860.

In some embodiments, and as best shown in FIG. 18C, the structurally rigid portion 1811 is thicker or has a greater height than the first flexible portion 1810a and second flexible portion 1810b. In this regard, the thickness or greater height provides further support and space for the sensor electronics 1860 (not illustrated) distributed or housed therein. In some embodiments, and as best shown in FIGS. 18A-18B, the structurally rigid portion 1811 can comprise a greater width than the first flexible portion 1810a and the second flexible portion 1810b. In some embodiments, the sensor control device 8102 comprises a uniform thickness and height. In some embodiments, the sensor electronics 1860 can be distributed across an entire area of the sensor control device 8102. As such, the sensor electronics 1860 (not illustrated) can be housed within the flexible portion(s) 1810 and/or the structurally rigid portion 1811. In some embodiments, the sensor control device 8102 does not include a structurally rigid portion.

According to an aspect of the embodiments, and as best shown in FIG. 18B, an adhesive 1824 is positioned on or otherwise attached to the underside 1890 of the sensor control device 8102. In this regard, the adhesive 1824 can be configured to secure and maintain the sensor control device 8102 in position on the user's skin surface during operation. Though not illustrated, in some embodiments, the adhesive 1824 can comprise a plurality of openings, such as pinholes or cone-shaped micro-holes, with a thin superabsorbent layer proximal relative thereto. The superabsorbent layer can include various materials and/or agents. For example, the superabsorbent layer can be composed of a thin desiccant layer, or other material configured to actively remove moisture and/or liquid. The superabsorbent layer can be configured so as to remove moisture and/or physical fluid from the skin of the user, such as sweat. Further, the plurality of openings in the adhesive 1824 allow fluid to travel in one direction but not in a second direction due to capillary action. In this regard, the openings or micro-holes are configured to absorb moisture and/or liquid being released from the sensor control device 8102. Further, the absorbed moisture is then absorbed by the superabsorbent layer so as to allow the adhesive 1824 to more effectively remain adhered to the skin surface of the user. Though not illustrated, in some embodiments, a moisture-barrier layer or backing layer is further provided on the top exterior surface 1880 (see, e.g., FIG. 18A) of the sensor control device 8102. Specifically, the backing layer (not illustrated) is configured to inhibit or prevent the superabsorbent layer from absorbing the moisture and/or liquid from the external environment. In this regard, the backing layer inhibits or prevents the moisture and/or liquid from the external environment from consuming the superabsorbent layer's absorbence capacity.

According to another aspect of the embodiments, the sensor control device 8102 can be similar in some respects to the sensor control device 102 of, for example, FIGS. 9A-9B, and therefore can be best understood with reference thereto. Specifically, the sensor control device 8102 can house an analyte sensor 8104 (see, e.g., FIG. 18B) integrated with sensor electronics 1860 (not shown). In some embodiments, and as best illustrated in FIG. 18B, the sensor control device 8102 can include one or more analyte sensors 8104. Specifically, the sensor control device 8102 can include an array of a plurality of analyte sensors 8104, wherein at least a portion of each analyte sensor 8104 is arranged within the electronics housing 1860 (FIG. 18C), and wherein each analyte sensor 8104 includes a sensor tail 8004 extending distally from the underside 1890 of the sensor control device 8102 (best shown in FIG. 18B), e.g., a bottom portion of the structurally rigid portion 1811 forming the electronics housing 1860. More specifically, the sensor control device 8102 can include an array of small, short, rigid, and sharp dermal analyte sensors or micro-analyte sensors 8104 (also herein referred to as “micro-sensor(s) 8104”). In this regard, the array of micro-sensors 8104 will allow for better fault checks and accuracy through averaging sensed analyte data.

As best illustrated in FIG. 18B, the array of micro-sensors 8104 can be disposed so as to protrude from the underside 1890 of the sensor control device 8102. In some embodiments, the array of micro-sensors 8104 can be disposed along the structurally rigid portion 1811 and at least a portion of the first flexible portion 1810a and second flexible portion 1810b (best shown in FIG. 18B).

According to some embodiments, the micro-sensors 8104 can be between one millimeter and two millimeters in length. In some embodiments, the micro-sensors can be any length less than five millimeters in length. In some embodiments, the micro-sensors 8104 can comprise a shortened sensor tail 8004 which forms a tip portion 8005 sufficiently sharpened so as to effectively penetrate the skin. The shortened sensor tail 8004 can be any length needed to reach an interstitial fluid of the user. In some embodiments, the shortened sensor tail can be between 0.8 millimeters (mm) to 3 mm in length. This sensor design can be advantageous in that it would remove the need for a sharp. Further, shorter analyte sensors, such as the micro-sensors 8104, will cause less trauma and could improve or eliminate the risk of early signal attenuation (“ESA”) created by wound trauma.

In some embodiments, the tail 8004 of each micro-sensor 8104 in the array can include an enzyme or other chemistry or biological and, in some embodiments, a membrane can cover the chemistry. In use, the tail 8004 is transcutaneously received beneath a user's skin. In some embodiments, the tail 8004 of each micro-sensor 8104 can include the same or different enzyme, chemistry, or biological composition as the other tails 8004 of each micro-sensor 8104 in the array. In some embodiments, the sensor tail 8004 is coated with dexamethasone so as to prolong the life of the micro-sensor 8104. Dexamethasone can utilize a control release mechanism and be used to prevent signal loss associated with shallower penetration depth of the shortened sensor tail 8004 and/or possible microphage attack of the inserted sensor tail 8004. In some embodiments, the sensor tail 8004 comprises an enteric coating composed of a material sufficiently sharp so as to remove the need for a sharp. Specifically, the enteric coating is configured so as to be hard enough to penetrate the user's skin but also change depending on the environment. For example, in some embodiments, the enteric coating on the tail 8004 of each micro-sensor 8104 is configured to soften and dissolve after the micro-sensor 8104 has been inserted into the user's skin. In this regard, once the enteric coating has dissolved, the chemistry on the tail 8004 of the micro-sensor 8104 can help facilitate analyte monitoring in the presence of bodily fluids.

According to an aspect of the embodiments, the sensor control device 8102 can comprise sensor electronics 1860 (not illustrated) which can include, a printed circuit board, one or more processors, one or more batteries, an antenna, a semiconductor chip, to name a few. In some embodiments, the sensor control device can include sensor electronics 1860 similar to the sensor electronics 160 described with reference to FIGS. 2B and 2C, or, e.g., 9A through 14. In some embodiments, the sensor control device 8102 can include one or more printed batteries 1899 (not illustrated). Specifically, the sensor control device 8102 can include stacks of multiple layers of printed batteries 1899 (not illustrated) so as to better manage battery life for the sensor control device 8102. According to an aspect of the embodiments, the printed batteries 1899 (not illustrated) are flexible.

Additionally, the printed circuit board can be made from fiberglass-reinforced epoxy-laminated sheets (e.g., FR4). In some embodiments, the printed circuit board can comprise a flexible material such that the printed circuit board can be folded or deformed while maintaining electrical communication with the componentry coupled thereto.

In some exemplar embodiments, a secondary device or external charging device can be utilized to manage the one or more batteries of the sensor control device 8102. In some embodiments, a secondary device can be utilized to offload some of the necessary sensor electronics componentry so as to minimize space needed for sensor electronics 1860 (not shown) disposed within the sensor control device 8102. In some embodiments, wherein sensor electronics 1860 are offloaded to a secondary device, the sensor control device 8102 can include sensor electronics 1860, such as, analog front end circuitry and the antenna. Further, the sensor control device 8102 can utilize wireless protocols which include BLE, BTLE, NFC, and others.

FIG. 18D depicts a top perspective view of an additional exemplar embodiment of a sensor control device 6102. Sensor control device 6102 is similar to sensor control device 8102 except that the structurally rigid portion 6111 comprises a width that is smaller than a width of the flexible portion(s) 6110. Specifically, the structurally rigid portion 6111 is rectangular in shape and protrudes in a proximal direction from the top exterior surface 1680 of the sensor control device 6102 so as to have a height greater than a height of the flexible portion(s). Further, the sensor control device 6102 is “band-aid” shaped and comprises a strip with rounded edges.

In some embodiments, the sensor control device 6102 can include an array of micro-sensors 6104 disposed along the flexible portion 6110 and structurally rigid portion 6111, as depicted in FIG. 18E. As shown in FIG. 18E, the array of micro-sensors 6104 can be arranged along a straight line. In other embodiments, the array of micro-sensors 6104 are disposed along the structurally rigid portion 6111 of the sensor control device 6102, as shown in FIG. 18F. As shown in FIG. 16F, the array of micro-sensors 6104 can be arranged in rows.

FIG. 18G depicts a top view of an additional exemplar embodiment of a sensor control device 7102. Sensor control device 7102 is similar to sensor control device 8102 except that the structurally rigid portion 7111 comprises a width that is smaller than a width of the flexible portion(s) 7110. Further, the sensor control device 7102 is circular in shape.

FIG. 18H depicts a bottom perspective view of an additional exemplar embodiment of a sensor control device 9102. Sensor control device 9102 is similar to sensor control device 8102 except that it is oval in shape.

In some embodiments, the sensor control devices 6102, 7102, 8102, and 9102 described herein can be applied to a user's skin surface without the use of an applicator. For example, the sensor control device 6102, 7102, 8102, or 9102 can be manually applied to the skin surface of the user. In some embodiments, the sensor control device 6102, 7102, 8102, or 9102 is initially assembled or rolled into a cylindrical shape and is configured to flatten and adhere to the skin surface as the user incrementally applies, dispenses, or rolls out the sensor control device 6102, 7102, 8102, or 9102 onto the skin surface. In this manner, the array of micro-sensors 6104, 7104, 8104, or 9104 are inserted into the user's skin as they make contact with the skin surface during the dispensing process or roll-out application process.

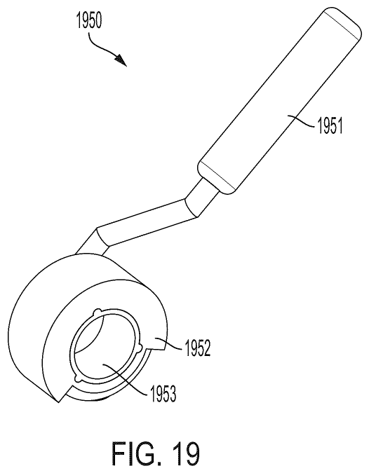

In other embodiments, the sensor control devices 6102, 7102, 8102, and 9102 can be used in conjunction with an applicator 1950 (as shown in FIG. 19), which can deliver the sensor control device 6102, 7102, 8102, or 9102 to a target monitoring location on a user's skin surface. Specifically, and as depicted in FIG. 19, the applicator 1950 can comprise a handle 1951 and a sensor control device dispenser 1952 (also herein referred to as a “dispenser”). More specifically, the handle 1951 can include a fixed dispenser receiving portion 1953 configured to securely hold the dispenser 1952 through the application process.

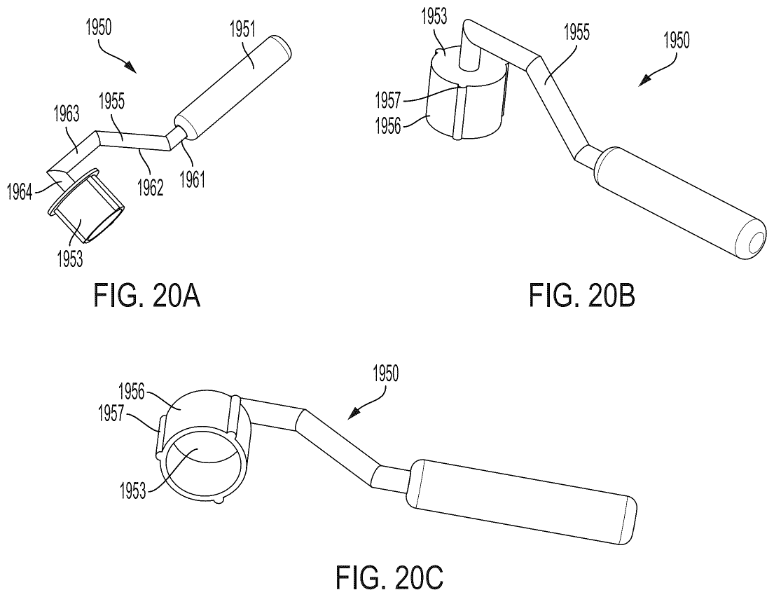

FIGS. 20A-20C depict an exemplar embodiment of the applicator 1950 without the dispenser 1952 disposed thereon. FIGS. 20A, 20B, and 20C, are side perspective, back side, and bottom side views, respectively, of the applicator 1950 comprising the handle 1951 and a cylindrical or substantially cylindrical shaped dispenser receiving portion 1953 with a hollow interior 1954. Specifically, the handle 1951 is configured to be held by the user and extends from a proximal portion of the applicator 1950. The handle 1951 can be an elongate handle 1951 with a generally cylindrical or barrel-like shape. Further, in some embodiments, a metal bar 1955 can extend from a distal end of the handle 1951, wherein the bottom end of the metal bar 1955 comprises the dispenser receiving portion 1953. In some embodiments, and as best shown in FIG. 20A, the metal bar 1955 is configured to comprise a first portion 1961, a second portion 1962, a third portion 1963, and a fourth portion 1964. Specifically, the first portion 1961 forms the top end of the metal bar 1955. More specifically, the fourth portion 1964 forms the bottom end, wherein the dispenser receiving portion 1953 extends longitudinally therefrom. Further, in some embodiments, the first portion 1961 lies along a different longitudinal axis than the fourth portion 1964. In some embodiments, the first portion 1961 is perpendicular or substantially perpendicular to the fourth portion 1964. In this regard, an exterior surface 1956 of the dispenser receiving portion 1953 is configured to be parallel to the user's skin surface when the user is holding the handle 1951 of the applicator 1950.

According to an aspect of the embodiments, and as best depicted in FIGS. 20B and 20C, the exterior surface 1956 of the dispenser receiving portion 1953 can include one or more locking features 1957 which are configured to interface with one or more corresponding slots 1987 on the dispenser 1952 so as to securely hold the dispenser 1952 (not shown in FIGS. 20A-20C) on the applicator 1950. In some embodiments, and as best shown in FIG. 20C, the dispenser receiving portion 1953 can include three locking features 1957.

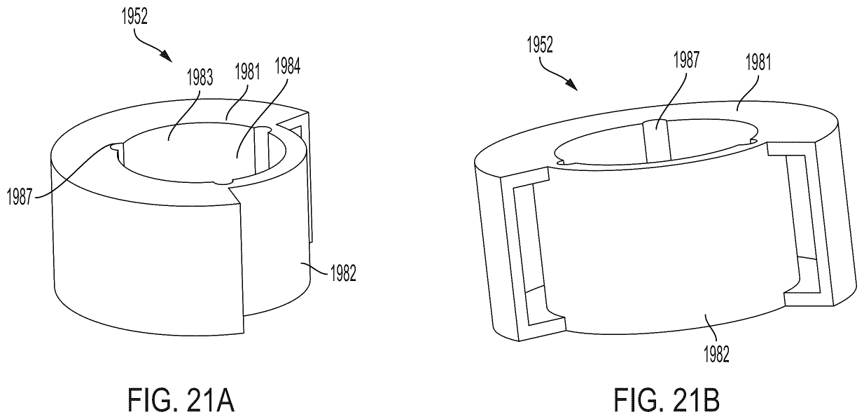

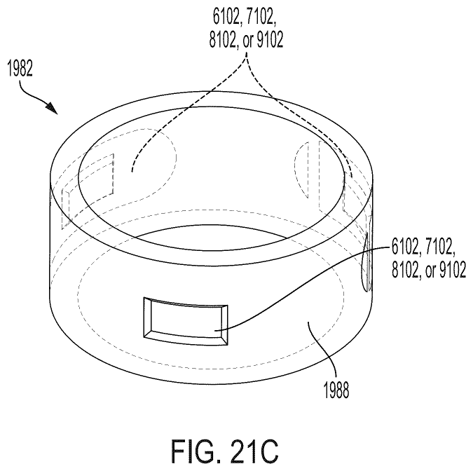

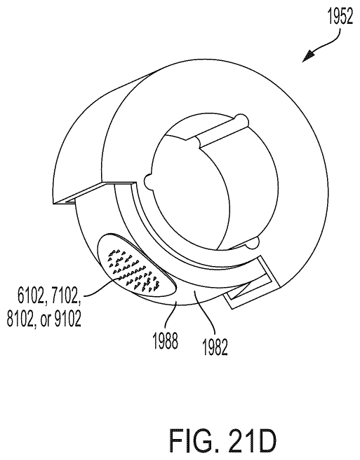

FIGS. 21A and 21B are top side perspective and side views, respectively, of the dispenser 1952. As shown in FIGS. 21A and 21B, the dispenser 1952 can comprise a shell 1981 and an inner ring 1982. Specifically, the shell 1981 is sized and configured to be received by the dispenser receiving portion 1953 (not depicted in FIGS. 21A-21B). More specifically, the shell 1981 is configured so as to be complementary in shape to the dispenser receiving portion 1953. In some embodiments, the shell 1981 can comprise a C-shape or substantially cylindrical shape. The shell 1981 is configured to partially enclose the inner ring 1982. Further, and as best shown in FIG. 21, the shell 1981 comprises an interior surface 1983 which defines an inner space 1984. According to an aspect of the embodiments, the interior surface 1983 of the shell 1981 has a diameter greater than a diameter of the exterior surface 1956 of the dispenser receiving portion 1953 (not shown in FIGS. 21A-21B) and is configured to interface therewith. Further, the one or more slots 1987 are arranged along the interior surface 1983 of the shell 1981. Thus, the one or more slots 1987 of the interior surface 1983 can engage with the one or more locking features 1957 of the dispenser receiving portion 1953 (not shown) as the interior surface 1983 of the shell 1981 interfaces with the exterior surface 1956 of the dispenser receiving portion 1953. Further, in some embodiments, the inner space 1984 is configured so as to align with the hollow interior 1954 of the dispenser receiving portion 1953 when the one or more slots 1987 are engaged with the one or more locking features 1957 (see, e.g., FIG. 19). In some embodiments, when the dispenser 1952 is received by the dispenser receiving portion 1953, it is configured to, e.g., pivot, rotate, or roll so as to dispense the sensor control device 6102, 7102, 8102, or 9102 during the application process. In some embodiments, a rotary mechanism is utilized to allow the dispenser 1952 to rotate about the dispenser receiving portion's 1953 axis. All moving components can be housed in the dispenser 1952.

FIG. 21C is a top side perspective view of the inner ring 1982 of the dispenser 1952. According to an aspect of the embodiments, one or more sensor control devices 6102, 7102, 8102, or 9102 can be arranged along an outer circumference 1988 of the inner ring 1982, as shown by the solid lines on the inner ring 1982 in FIG. 21C. In this regard, the inner ring 1982 can define a roller for the sensor control device(s) 6102, 7102, 8102, or 9102, as the sensor control device(s) 6102, 7102, 8102, or 9102 are configured to be released from the inner ring 1982 and adhere to the skin surface through the roll-out process utilizing the applicator 1950.

Specifically, and still with reference to FIG. 21C, the portion of the inner ring 1982 which is enclosed by the shell 1981 (not shown in FIG. 21C) can store one or more sensor control devices 6102, 7102, 8102, or 9102. In this regard, the shell 1981 can define a protective enclosure or cover for the stored one or more sensor control devices 6102, 7102, 8102, or 9102. Though not illustrated, in some embodiments, a dust cover and/or a moisture seal can be provided to protect the stored one or more sensor control devices 6102, 7102, 8102, or 9102. Further, the outer circumference 1988 of the inner ring 1982 defines a skin contacting surface. Specifically, a portion of the inner ring 1982 that is exposed or not enclosed by the shell 1981 is configured to interface with the skin surface of the user.

FIG. 21D is a side perspective view of the dispenser 1952 comprising the inner ring 1982 with a sensor control device 6102, 7102, 8102, or 9102 arranged thereon. Specifically, an underside of the sensor control device 6102, 7102, 8102, or 9102 is exposed and configured for placement on the user's skin surface. More specifically, an adhesive material (not depicted) can be disposed between the inner ring 1982 and the sensor control device(s) 6102, 7102, 8102, or 9102. In some embodiments, the adhesive material (not shown) is configured so as to more strongly adhere to the outer circumference 1988 of the inner ring 1982 than the sensor control device 6102, 7102, 8102, or 9102. In this regard, as the sensor control device is released from the inner ring 1982, the adhesive material (not shown) remains on the inner ring 1988 and does not transfer with the sensor control device 6102, 7102, 8102, or 9102.

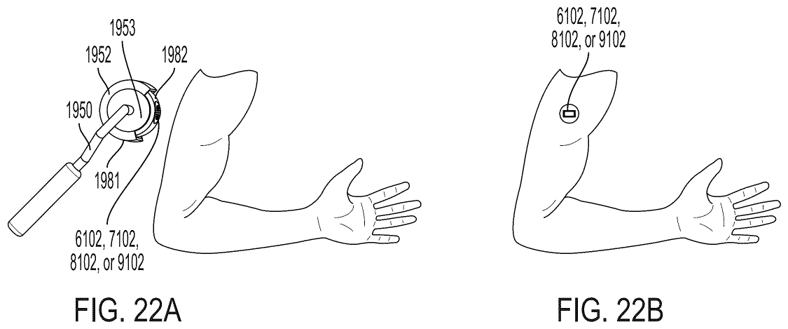

FIGS. 22A and 22B illustrate various stages of the dispensing process or roll-out application process with the applicator 1950. With reference to FIG. 22A, to apply a sensor control device 6102, 7102, 8102, or 9102 onto the user's skin surface, the dispenser 1952 must be assembled onto the handle's dispenser receiving portion 1953 and, subsequently, dispensed or rolled across an insertion site. The sensor control device 6102, 7102, 8102, or 9102 that will be applied to the user's skin surface is not initially visible and is rather enclosed by the shell 1981 prior to application. Specifically, to expose the sensor control device 6102, 7102, 8102, or 9102 that will be applied to the user's skin surface, the inner ring 1982 must be rotated via, e.g., rolling. According to some embodiments, the user must first exert a downward pressure on the handle 1951 to initiate the dispensing process or roll-out application process Specifically, a minimum insertion pressure can be achieved by ensuring that dispensing or rolling does not activate until a predetermined amount of pressure is manually applied by the user. In this regard, the application process can require a “push and roll” mechanism. Once the application process has been initiated by the minimum insertion pressure being achieved, the user can then roll the dispenser 1952 so as to apply the sensor control device 6102, 7102, 8102, or 9102 onto the user's skin surface, as shown in FIG. 22B.

In some embodiments, a detent (not shown) is used to initiate the rolling of the dispenser 1952 until an audible and/or tactile feedback (e.g., a click) is experienced. The audible and/or tactile feedback is configured to indicate to the user that one sensor control device 6102, 7102, 8102, or 9102 has been successfully dispensed from the dispenser 1952 and applied onto the skin surface of the user. Specifically, the sensor control device 6102, 7102, 8102, or 9102 is configured to roll off or dispensed from the dispenser 1952 and adhere to the user's skin surface as the inner ring 1982 rotates and completes one full roll cycle. In some embodiments, the dispenser 1952 comprises a mechanism that causes the user to complete the full roll cycle by forcing the rolling movement to continue until the audible and/or tactile feedback is experienced. In this manner, the mechanism prevents the user from prematurely ceasing the application process or stopping midway so as to not entirely dispense the sensor control device 6102, 7102, 8102, or 9102 from the dispenser 1952.

In some embodiments, the dispenser 1952 is designed so as to be held directly by the user without the need for the handle 1951. For example, the user can manually hold the dispenser 1952 loaded with the sensor control device(s) 6102, 7102, 8102, or 9102, as shown in FIG. 21D, and proceed with the push and roll mechanism so as to apply the sensor control device 6102, 7102, 8102, or 9102. Specifically, the user can apply a downward force onto the dispenser 1952 to initiate application, and a detent (not shown) can be utilized to initiate the dispensing or rolling process so as to dispense the sensor control device 6102, 7102, 8102, or 9102 onto the skin surface.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

Exemplary embodiments are set forth in the following numbered clauses;

1. A system for measurement of an analyte level, comprising:
an analyte sensor integrated with one or more sensor electronics, the analyte sensor comprising an in vivo portion and an ex vivo portion, wherein the ex vivo portion comprises an aperture from which the in vivo portion extends, wherein the in vivo portion is configured to be in contact with an interstitial fluid of a user, wherein the one or more sensor electronics are mounted on at least one surface of the ex vivo portion;
a sensor control device configured to house the analyte sensor integrated with the one or more sensor electronics;
a sharp carrier comprising a sharp, wherein a portion of the sharp is configured to engage with a portion of the sharp carrier, and wherein the sharp is configured to place the in vivo portion of the analyte sensor in contact with the interstitial fluid of the user.

2. The system of clause 1, wherein the one or more sensor electronics comprises an NFC antenna.

3. The system of clause 1 or 2, wherein the sensor control device comprises a housing, wherein the housing comprises an upper portion and a mount portion, and wherein the upper portion and mount portion form a unitary piece.

4. The system of clause 1, 2 or 3, wherein the aperture of the ex vivo portion comprises a space configured for receipt of the in vivo portion, wherein the in vivo portion is configured to extend from the space and is positioned centrally relative to the ex vivo portion.

5. The system of any of clauses 1 to 4, wherein the analyte sensor is configured to fold at a neck, wherein the neck interconnects the ex vivo portion and the in vivo portion.

6. The system of any preceding clause, wherein the one or more sensor electronics comprises a battery, wherein the battery comprises a first tab and a second tab, wherein the first tab is oriented radially and coplanar to the second tab.

7. The system of clause 6, wherein the first tab comprises a first base that is configured to be welded on a positively charged surface of the battery.

8. The system of any preceding clause, wherein the one or more sensor electronics comprises a battery, wherein the battery comprises a first tab and a second tab, wherein the first tab is parallel and symmetric to the second tab.

9. The system of clause 8, wherein the first tab comprises a first base that is configured to be welded on a positively charged surface of the battery.

10. The system of clause 8 or 9, wherein the second tab comprises a second base that is configured to be welded on an edge portion of the battery.

11. The system of any preceding clause, wherein at least a portion of the ex vivo portion is folded.

12. The system of clause 11, wherein the analyte sensor comprises a neck having a single fold, wherein the one or more sensor electronics comprises a folded antenna and a battery, wherein at least a portion of the battery is hanging from a periphery of the ex vivo portion.

13. The system of clause 11, wherein the analyte sensor comprises a neck having a double fold, wherein the one or more sensor electronics comprises a folded antenna and a battery, wherein at least a portion of the battery is hanging from a periphery of the ex vivo portion.

14. The system of any preceding clause, wherein the sensor control device is configured to undergo low energy terminal e-beam sterilization.

15. The system of any preceding clause, wherein the sharp comprises a cantilever arm, wherein the cantilever arm is configured to engage with a portion of the sharp carrier.

16. The system of any preceding clause, wherein the sharp comprises a pair of sharp tabs configured to mate with an upper portion of a sensor control device.

17. The system of any preceding clause, wherein the sensor control device comprises an upper portion and a mount portion, wherein an aperture extends through the upper portion and mount portion, wherein at least a portion of the sharp is configured to extend through the aperture extending through the upper portion and mount portion.

18. The system of any preceding clause, wherein the sharp carrier comprises an inner portion with a cavity, wherein the cavity is configured to receive an upper portion of the sharp.

19. The system of any preceding clause, wherein the sharp carrier comprises an inner portion and a hollow outer portion, wherein the hollow outer portion comprises a first diameter and the inner portion comprises a second diameter, wherein the first diameter is larger than the second diameter.

20. The system of any preceding clause, wherein the sharp comprises a sensor channel and a distal tip, wherein the sensor channel is configured to receive the in vivo portion of the analyte sensor, and wherein the sensor channel is in a spaced relation to the distal tip.

21. The system of any preceding clause, wherein the sensor control device comprises a housing, wherein at least a portion of the analyte sensor is disposed within the housing.

22. The system of any preceding clause, wherein the ex vivo portion further comprises a first surface and a second surface, wherein the first surface comprises a battery, and wherein the second surface comprises an antenna.

23. The system of any preceding clause, wherein the analyte sensor and the one or more sensor electronics are formed from a one-piece substrate.

24. The system of clause 23, wherein the substrate can be formed through a die-cutting process, a laser cutting process, an ultrasonic cutting process, a molding process, a stamping process, or a 3-D printing process.

25. The system of clause 23 or 24, wherein the substrate is made from a flexible non-electrically-conductive polymer.

26. The system of clause 23, 24, or 24, wherein the substrate is a polyamide substrate, a polyester substrate, or a polyethylene terephthalate substrate.

27. A system for measurement of an analyte level, comprising:
a flexible sensor control device configured to house one or more sensor electronics;
an array of a plurality of analyte sensors disposed along the sensor control device, wherein each of the plurality of analyte sensors comprises a sensor tail configured to be inserted under a skin surface of a user; and
an applicator configured to apply the sensor control device onto the skin surface of the user, wherein the applicator comprises a dispenser configured to be dispensed, and wherein upon the dispenser being dispensed, the applicator is configured to apply the sensor control device onto the skin surface.

28. The system of clause 27, wherein the sensor control device is partially or entirely flexible, and wherein the sensor control device is configured to contour to a body of the user.

29. The system of clause 27 or 28, wherein the sensor control device comprises a structurally rigid portion, a first flexible portion, and a second flexible portion, and wherein the structurally rigid portion houses the one or more sensor electronics.

30. The system of clause 27, 28, or 29, wherein the sensor control device comprises a structurally rigid portion and one or more flexible portions, wherein the one or more sensor electronics are housed within the structurally rigid portion and the one or more flexible portions.

31. The system of any of clauses 27 to 30, wherein the sensor tail of each of the plurality of analyte sensors is a shortened sensor tail comprising a sharpened tip portion, wherein the shortened sensor tail is between 0.8 millimeters and three millimeters in length.

32. The system of any of clauses 27 to 31, further comprising an adhesive positioned on an underside of the sensor control device, wherein the adhesive is configured to secure and maintain the sensor control device in position on the user's skin surface.

33. The system of any of clauses 27 to 32, further comprising an adhesive, wherein the adhesive comprising a plurality of openings configured to absorb moisture and liquid released from the sensor control device.

34 The system of clause 33, further comprising a superabsorbent layer configured to absorb moisture and liquid absorbed by the plurality of openings.

35 The system of clause 34, further comprising a backing layer configured on a top exterior surface of the sensor control device, wherein the backing layer is further configured to inhibit the superabsorbent layer from absorbing moisture and liquid from an external environment.

36. The system of any of clauses 27 to 35, wherein the one or more sensor electronics comprises one or more printed batteries, wherein each of the one or more printed batteries are configured to be flexible.

37. The system of any of clauses 27 to 36, wherein the one or more sensor electronics comprises a stack of multiple layers of printed batteries.

38 The system of any of clauses 27 to 37, wherein the sensor control device comprises a structurally rigid portion, a first flexible portion, and a second flexible portion, wherein the structurally rigid portion is disposed between the first flexible portion and the second flexible portion.

39. The system of any of clauses 27 to 38, wherein the sensor control device comprises a structurally rigid portion and one or more flexible portions, wherein the structurally rigid portion comprises a thickness that is greater than a thickness of the one or more flexible portions.

40. The system of any of clauses 27 to 39, wherein the sensor control device is a flexible strip with rounded edges.

41. The system of any of clauses 27 to 40, wherein the sensor tail of each of the plurality of analyte sensors is coated with dexamethasone, wherein the dexamethasone is configured to inhibit signal loss and utilizes a control release mechanism.

42. The system of any of clauses 27 to 41, wherein the sensor tail of each of the plurality of analyte sensors comprises an enteric coating configured to dissolve after insertion of the sensor tail.

43. The system of any of clauses 27 to 42, wherein the applicator comprises a handle and a dispenser receiving portion configured to receive the dispenser.

44. The system of any of clauses 27 to 43, wherein the dispenser is configured to hold one or more sensor control devices.

45 The system of any of clauses 27 to 44, wherein the dispenser comprises a shell and an inner ring, wherein the sensor control device is arranged along the inner ring.

46. The system of any of clauses 27 to 45, wherein the dispenser can store one or more sensor control devices, wherein the dispenser comprises a shell and an inner ring, wherein the shell is configured to partially enclose the inner ring, and wherein the shell can form a protective cover for the stored one or more sensor control devices.

47. The system of any of clauses 27 to 46, wherein the applicator is configured to provide an audible or tactile feedback to indicate that the sensor control device has been successfully applied to the user's skin surface.

48. The system of any of clauses 27 to 47, wherein each of the plurality of analyte sensors comprises a sensor tail extending distally from the underside of the sensor control device.

1-26. (canceled)

27. A system for measurement of an analyte level, comprising:
a flexible sensor control device configured to house one or more sensor electronics;
an array of analyte sensors disposed along a distal-facing surface of the sensor control device, wherein each analyte sensor in the array of analyte sensors comprises a distal portion configured to be positioned under a skin surface of a user; and
an applicator configured to apply the sensor control device onto the skin surface of the user, wherein the applicator comprises a dispenser.

28. The system of claim 27, wherein the sensor control device is partially or entirely flexible, and wherein the sensor control device is configured to contour to a body of the user.

29. The system of claim 27, wherein the sensor control device comprises a structurally rigid portion, a first flexible portion, and a second flexible portion, and wherein the structurally rigid portion houses the one or more sensor electronics.

30. The system of claim 27, wherein the sensor control device comprises a structurally rigid portion and one or more flexible portions, wherein the one or more sensor electronics are housed within the structurally rigid portion and the one or more flexible portions.

31. The system of claim 27, wherein the distal portion of each analyte sensor in the array of analyte sensors is a shortened distal portion comprising a sharpened tip portion, wherein the shortened distal portion is between 0.8 millimeters and three millimeters in length.

32. The system of claim 27, further comprising an adhesive positioned on an underside of the sensor control device, wherein the adhesive is configured to secure and maintain the sensor control device in position on the user's skin surface.

33. The system of claim 27, further comprising an adhesive, wherein the adhesive comprises a plurality of openings configured to absorb moisture and liquid released from the sensor control device.

34. The system of claim 33, further comprising a superabsorbent layer configured to absorb moisture and liquid absorbed by the plurality of openings.

35. The system of claim 34, further comprising a backing layer configured on a top exterior surface of the sensor control device, wherein the backing layer is further configured to inhibit the superabsorbent layer from absorbing moisture and liquid from an external environment.

36. The system of claim 27, wherein the one or more sensor electronics comprises one or more printed batteries, wherein each of the one or more printed batteries are configured to be flexible.

37. The system of claim 27, wherein the one or more sensor electronics comprises a stack of multiple layers of printed batteries.

38. The system of claim 27, wherein the sensor control device comprises a structurally rigid portion, a first flexible portion, and a second flexible portion, wherein the structurally rigid portion is disposed between the first flexible portion and the second flexible portion.

39. The system of claim 27, wherein the sensor control device comprises a structurally rigid portion and one or more flexible portions, wherein the structurally rigid portion comprises a thickness that is greater than a thickness of the one or more flexible portions.

40. The system of claim 27, wherein the sensor control device is a flexible strip with rounded edges.

41. The system of claim 27, wherein the distal portion of each analyte sensor in the array of analyte sensors is coated with dexamethasone, wherein the dexamethasone is configured to inhibit signal loss and utilizes a control release mechanism.

42. The system of claim 27, wherein the distal portion of each analyte sensor in the array of analyte sensors comprises an enteric coating configured to dissolve after insertion of the sensor tail.

43. The system of claim 27, wherein the applicator comprises a handle and a dispenser receiving portion configured to receive the dispenser.

44. The system of claim 27, wherein the dispenser is configured to hold one or more sensor control devices.

45. The system of claim 27, wherein the dispenser comprises a shell and an inner ring, wherein the sensor control device is arranged along the inner ring.

46. The system of claim 27, wherein the dispenser can store one or more sensor control devices, wherein the dispenser comprises a shell and an inner ring, wherein the shell is configured to partially enclose the inner ring, and wherein the shell can form a protective cover for the stored one or more sensor control devices.

47. The system of claim 27, wherein the applicator is configured to provide an audible or tactile feedback to indicate that the sensor control device has been successfully applied to the user's skin surface.

48. The system of claim 27, wherein each analyte sensor in the array of analyte sensors comprises a distal portion extending distally from the underside of the sensor control device.
